(19) United States
(12) Patent Application Publication
(10) Pub. No.: US 2022/0194939 A1
(43) Pub. Date: Jun. 23, 2022

(54) Novel compounds and methods of use treating fructose-related disorders or diseases

(71) Applicants: Colorado Research Partners, LLC, Centennial, CO (US); The Regents of the University of Colorado, A Body Corporate, Denver, CO (US)

(72) Inventors: Sundeep Dugar, Centennial, CO (US); Chase Needham, Wilmington, NC (US); Paul Maffuid, Carlsbad, CA (US); Rajendra Kumar Reddy Gadikota, Wilmington, NC (US); Sreenivasulu Reddy Koduru, Wilmington, NC (US); Michael F. Wempe, Aurora, CO (US); Vijay Kumar, Aurora, CO (US)

(21) Appl. No.: 17/603,988

(22) PCT Filed: Apr. 17, 2020
(86) PCT No.: PCT/US2020/028865

§ 371 Date: Oct. 15, 2021

Related U.S. Application Data
(60) Provisional application No. 62/835,015, filed on Apr. 17, 2019.

(51) Int. Cl. C07D 471/04; C07D 231/56; A61K 45/06; C07D 403/12; C07D 401/12; C07D 249/18

(52) U.S. Cl. CPC C07D 471/04; C07D 231/56; C07D 249/18; C07D 403/12; C07D 401/12; A61K 45/06

(57) ABSTRACT

Disclosed herein are novel compounds that inhibit fructokinase (KHK or ketohexokinase) and the downstream metabolic effects mediated by fructose metabolism. Fructokinase inhibitors specifically block the metabolism of both dietary and endogenous fructose metabolism and have a host of potential metabolic benefits.

### CROSS REFERENCE TO RELATED APPLICATIONS

This application claims priority to and the benefit of U.S. Provisional Application No. 62/835,015, filed Apr. 17, 2019, the entirety of which is hereby incorporated by reference.

### FEDERAL FUNDING

This application was made with government support awarded by the National Institutes of Health (Award Nos. NIH R42 DK104432-03, 5R42DK104432-03, and 1U01AA027997-01). The Government of the United States has certain rights in the invention.

### FIELD

This disclosure relates generally to compounds that inhibit fructokinase (aka ketohexokinase) and the downstream metabolic effects mediated by fructose metabolism.

### BACKGROUND

Fructokinase (ketohexokinase, KHK) is a key enzyme in fructose metabolism, and phosphorylates fructose to fructose-1-phosphate. In turn, fructose 1-phosphate is metabolized by aldolase B and triokinase to dihydroxyacetone phosphate and glyceraldehyde 3-phosphate, which leads eventually to glycolysis and the generation of triglycerides. There are two isoforms of fructokinase, namely fructokinase C and fructokinase A. Fructokinase C is a rapid phosphorylator, and causes a transient fall in intracellular ATP and phosphate during the metabolism of fructose¹.

Studies show that the fructokinase C isoform is responsible for how fructose induces metabolic syndrome, fatty liver, cardiovascular and renal disease^{1,2}. Blocking fructokinase therefore may be a strategy in treating these conditions. In addition, blocking fructokinase activity may protect against sugar craving, sugar-induced hunger, and impaired satiety from leptin resistance. With respect to fructokinase A, fructokinase A may have a role in hepatic carcinoma³. While much of human exposure to fructose comes from dietary sources of fructose, such as table sugar (sucrose), high fructose corn syrup (HFCS), honey, and fruits, fructose can also be generated endogenously from glucose via the aldose reductase pathway. The aldose reductase pathway may play an important role as the mechanism by which high glycemic carbohydrates and salt induce metabolic syndrome and fatty liver, how heat stress and dehydration cause kidney disease, and also how diabetes mediates some of its complications, including renal disease and fatty liver⁴⁻⁶.

As well, Hereditary Fructose Intolerance (HFI) is a rare hereditary disease caused by aldolase B deficiency, the enzyme that metabolizes fructose-1-phosphate (the enzymatic step after fructokinase). The disease is characterized by rapid clinical symptoms in response to fructose ingestion, including hypoglycemia and lactate generation, with long-term manifestations including chronic liver disease. Fructokinase inhibition will also block the clinical manifestations of HFI in response to fructose.

To date, a number of KHK inhibitors have been uncovered which have been found to have activity in treating or preventing KHK-related or fructose-related disorders. See, for example, (i) US 20130224218, entitled “Methods and Compositions for the Inhibition of Fructokinase;” (ii) WO2012019188, entitled “Methods and Compositions for the Inhibition of Fructokinase; and (iii) WO2018170517, entitled “Indazole Inhibitors of Fructokinase (KHK) and Methods of Use in Treating KHK-Mediated Disorders or Diseases.” If fructose metabolism (whether starting from ingested or endogenously generated fructose) can be blocked or otherwise regulated by such inhibitors, compounds shown to have ability to inhibit fructokinase or other components of the fructose metabolism pathway are expected to continue to be suitable for treating fructose-related diseases or disorders, e.g., KHK-mediated diseases or disorders discussed above. While a number of KHK inhibitors have been developed for these purposes, there remains a significant need for KHK inhibitors with increased efficacy and selectivity for the associated target disease or disorder.

### BRIEF SUMMARY

The present application discloses a plurality of compounds that inhibit fructokinase (also referred to as ketohexokinase or KHK) and the downstream metabolic effects mediated by fructose metabolism (referred to also as “fructokinase or KHK inhibitors” herein). Various embodiments provide fructokinase inhibitors that specifically block both the metabolism of both dietary and endogenous fructose metabolism and have a host of potential metabolic benefits. These benefits may include, but are not limited to blocking sugar craving, as well as sugar induced metabolic syndrome, diabetes and fatty liver. Additionally, blocking fructose metabolism via the disclosed fructokinase inhibitors can benefit the rare orphan disease of Hereditary Fructose Intolerance, obesity, insulin resistance, metabolic syndrome, liver disease (including alcohol-related liver disease (ALD) and non-alcoholic fatty liver disease (NAFLD)), fatty liver, hypertension, cardiac injury from ischemia, certain cancers (including hepatocellular and pancreatic), acute kidney injury from ischemia, heat stress, rhabdomyolysis or radiocontrast, and/or chronic diabetic and nondiabetic renal disease.

### DETAILED DESCRIPTION

### Exemplary Compounds

In accordance with an aspect, there is disclosed herein compounds having a formula as set forth in Formula (I) below:

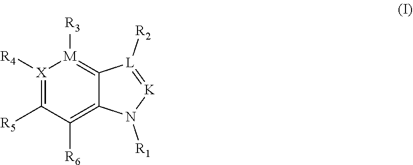

or a pharmaceutically acceptable salt, prodrug, or stereoisomer thereof,

wherein

M, L, K, and X are each independently=carbon or nitrogen;

R1=hydrogen, halogen, alkyl, ether, aryl, heteroaryl, cyclo, or heterocyclo,

R2=hydrogen, deuterium, halogen, cyano, alkyl, ether, cyclo, or heterocyclo when L=C or R2 is not present when L=nitrogen;

R3=hydrogen, halogen, deuterium, hydroxy, amino, alkyl, ether, alkoxy, cyano, cyclo, or heterocyclo when M=C or R3 is not present when M=nitrogen, wherein, when n=1 and m=1, R3=fluoro;

R4 and R6 are each independently=hydrogen, deuterium, halogen, alkyl, ether, cyclo, or heterocyclo;

R5=a substitution according to Formula (IIa) or (IIb):

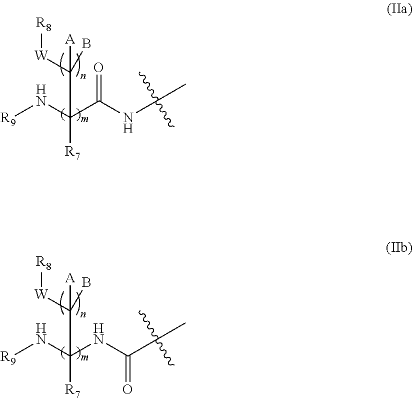

wherein R7 is selected from H, D, O-lower alkyl, or lower alkyl;

A and B are independently H, D, CH₃, CH₂CH₃, or cycloalkyl, or collectively define a carbonyl group, or are joined to form a 3-6 membered ring with a heteroatom or a heterofunctional group selected from O, NH, N-lower alkyl, S, or SO₂;

W is selected from CH₂, CHR7, C(R7)₂, O, or NH;

R8 is selected from H, —COAlkyl, —COAryl, —COOAlkyl, —COOAryl, —CONHAlkyl, or —CONHAryl;

R9 is selected from H, lower alkyl, —COOAlkyl, or —COOAryl;

n is 0, 1, 2, or 3;

m independently of n is 1, 2, or 3, wherein, when n=0, m>1;

wherein when K=carbon, then R1=m-tolyl or p-fluoro, R3=hydrogen or fluoro, and R5=serine,

wherein R7 and R9 are optionally joined to form a 4-7 membered ring, and wherein, when present, the 4-7 membered ring optionally comprises 0-5 methyl groups, OMethyl, or OR8; and

wherein, when m>1 and W=CH₂, W and R7 are optionally joined to form a 3-6 membered carbocyclic or heterocyclic ring comprising 0 or R9.

In an embodiment, the compound comprises one wherein R7 and R9 are joined to form a 4-7 membered ring, and the 4-7 membered ring comprises 0-5 methyl groups, OMe, or OR8, and in a particular embodiment, from 1-3 methyl groups.

In another embodiment, m>1 and W=CH₂, and W and R7 are joined to form a 3-6 membered carbocyclic or heterocyclic ring comprising 0 or R9.

In another embodiment, K=C such that the compound of Formula (I) comprises an indole core instead of an indazole, e.g., a compound having the formula of Formula (III):

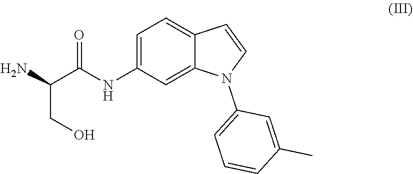 [SMILES: Cc1cccc(-n2ccc3ccc(NC(=O)[C@H](N)CO)cc32)c1]

wherein when K=carbon, R1=m-tolyl or p-fluoro, R3=hydrogen, and R5=serine,

R1 may be hydrogen, halogen, alkyl, ether, aryl, heteroaryl, cyclo, or heterocyclo as set forth above in Formula (I). In certain embodiments, R1=a moiety having the formula (IV):

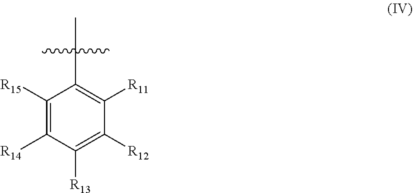

wherein, in Formula (IV), each of R11, R12, R13, R14, and R15 is independently selected from hydrogen, deuterium, halogen, hydroxy, a carboxylic acid moeity, amido, alkyl, aryl, heteroaryl, cyclo, heterocyclo, alkoxy, haloalkyl, haloalkoxy, or carboxylamido.

In particular embodiments, R11-R15 are each independently selected from —H, -D, —F, —Cl, Br, —CF₃, —CF₂H, —CFH₂, —OMe, —OCF₃, —C(O)NH₂, or —CH₃.

In further embodiments, R1=an aryl group selected from at least one of the following moieties:

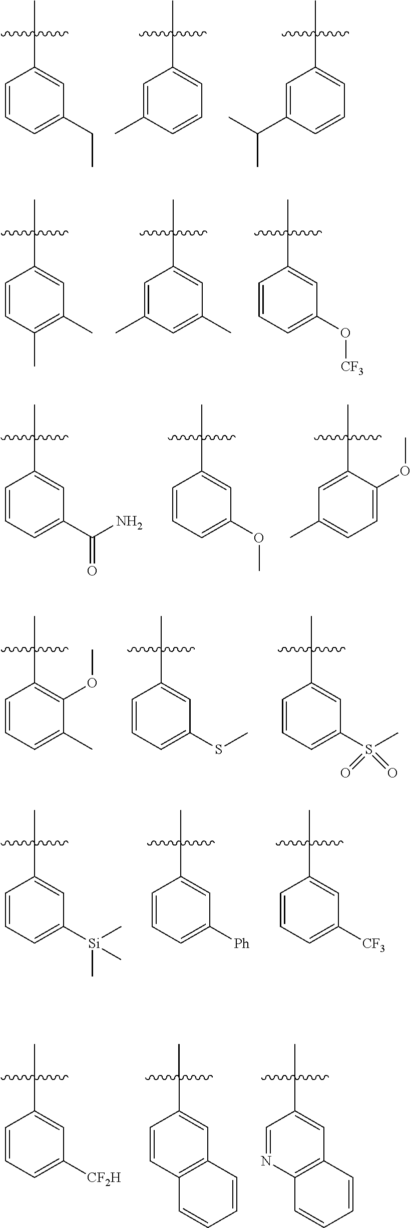

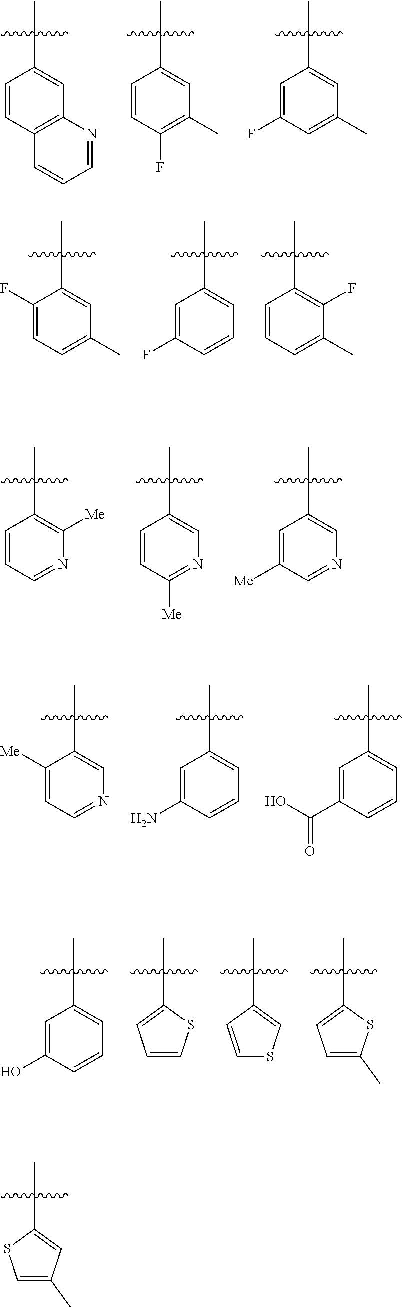

In an embodiment,

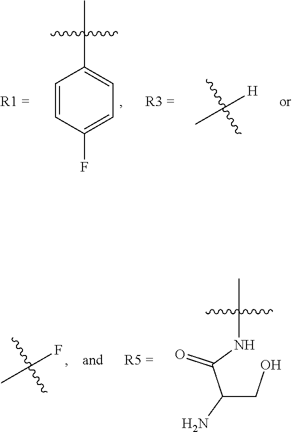

In another embodiment,

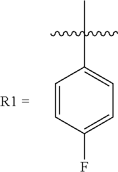 [SMILES: *c1ccc(F)cc1]

and R5=serine, and in a particular embodiment comprises a formula according to (Vb):

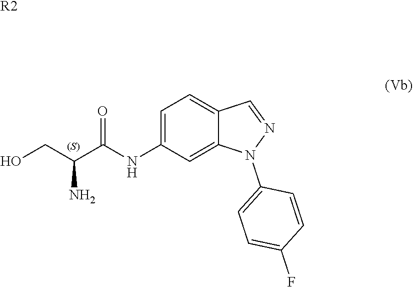

R2 may be hydrogen, deuterium, halogen, cyano, alkyl, ether, cyclo, or heterocyclo when L=C or R2 is not present when L=N. In certain embodiments, L=C and R2 is selected from at least one of the following moieties:

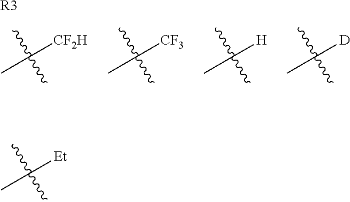

R3 may be hydrogen, halogen, deuterium, hydroxy, amino, alkyl, ether, alkoxy, cyano, cyclo, or heterocyclo when L=C or R3 is not present when L=N. In certain embodiments, L=C and R3 is selected from at least one of the following moieties:

—CH₃,

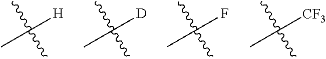

In an embodiment, M=N and R3 is not present, e.g., as in the compound of Formula (VI):

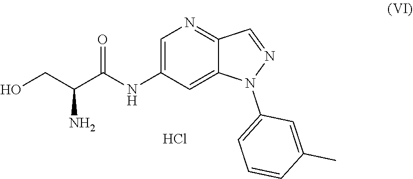 [SMILES: Cc1cccc(-n2ncc3ncc(NC(=O)[C@@H](N)CO)cc32)c1 ; Cl]

R4 and R6

R4 may be hydrogen, deuterium, halogen, alkyl, ether, cyclo, or heterocyclo.

Similarly, R6 may be hydrogen, halogen, alkyl, ether, cyclo, or heterocyclo. In certain embodiments, R4 and R6 are independently selected from at least one of the following moieties:

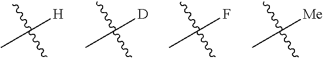

As noted above, R5=a substitution of Formula (IIa) or (IIb):

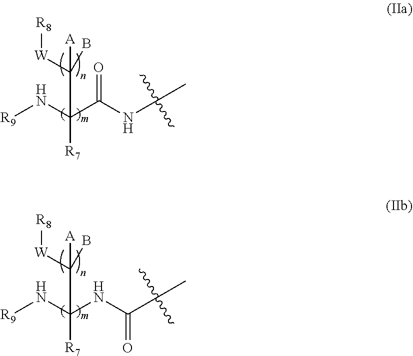

wherein R7 is selected from H, D, O-lower alkyl, or lower alkyl;

A and B are independently H, D, CH₃, CH₂CH₃, or cycloalkyl, or collectively define a carbonyl group, or are joined to form a 3-6 membered ring with a heteroatom or a heterofunctional group selected from O, NH, N-lower alkyl, S, or SO₂;

W is selected from CH₂, CHR7, C(R7)₂, O, or NH;

R8 is selected from H, —COAlkyl, —COAryl, —COOAlkyl, —COOAryl, —CONHAlkyl, or —CONHAryl;

R9 is selected from H, lower alkyl, —COOAlkyl, or —COOAryl;

n is 0, 1, 2, or 3;

m independently of n is 1, 2, or 3, wherein, when n=0, m>1;

wherein when K=carbon, R1=m-tolyl or p-fluoro, R3=hydrogen or fluoro, and R5=serine,

wherein R7 and R9 are optionally joined to form a 4-7 membered ring, and wherein, when present, the 4-7 membered ring optionally comprises 0-5 methyl groups, OMethyl, or OR8; and

wherein, when m>1 and W=CH₂, W and R7 are optionally joined to form a 3-6 membered carbocyclic or heterocyclic ring comprising O or R9.

In an embodiment, the compound comprises one wherein R7 and R9 are joined to form a 4-7 membered ring, and the 4-7 membered ring comprises 0-5 alkyl groups, OMe, or OR8, and in a particular embodiment, from 1-3 methyl groups.

In another embodiment, R7 and R9 are joined to form a 3-7 membered ring comprising 0-5 alkyl groups. In an embodiment, when present, the alkyl group(s) comprise methyl.

In yet another embodiment, m>1 and W=CH₂, and W and R7 are joined to form a 3-6 membered carbocyclic or heterocyclic ring comprising O or R9.

In certain embodiments, R5 comprises one of Formulas (VII)-(XV):

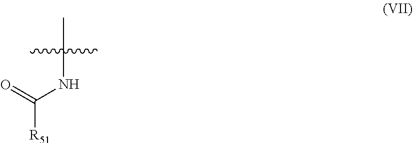

where R51=hydrogen, halogen, alkyl, ether, aryl, heteroaryl, cyclo, or heterocyclo;

or

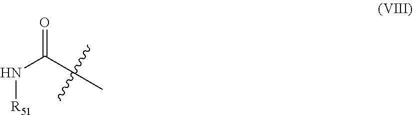

where R51=hydrogen, halogen, alkyl, ether, aryl, heteroaryl, cyclo, or heterocyclo; or

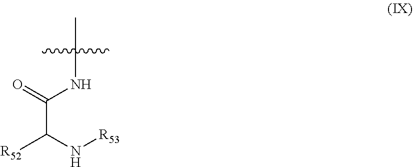

where R52 and R53 are independently hydrogen, halogen, alkyl, ether, aryl, heteroaryl, cyclo, or heterocyclo;

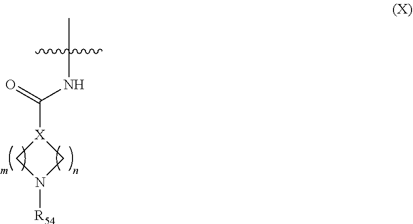

where n and m are independently=0 to 3 (and in a particular embodiment, m=1, n=1 and R3 in Formula (I)=F),

X in Formula (X)=C or N, and

R54=hydrogen, halogen, alkyl, ether, aryl, heteroaryl, cyclo, or heterocyclo; or

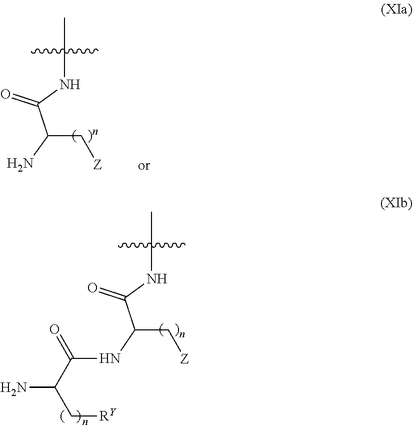

where n=0 to 5,

Z=alkyl, hydroxyl, —B(OH)₂, cyano, carboxyl, an amino acid functional group, alkylamino, alkylcarboxy, heterocyclic, aryl, heteroaryl, alkyl sulfide, thiol, or alkylurea; and

R_{Y}=alkyl, hydroxyl, —B(OH)₂, cyano, carboxyl, an amino acid functional group, alkylamino, alkylcarboxy, heterocyclic, aryl, heteroaryl, alkyl sulfide, thiol, or alkylurea; or

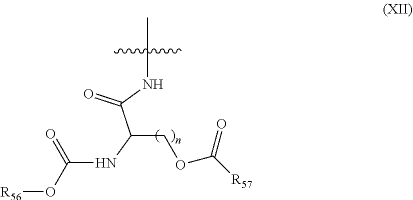

where n=0 to 5, and

R56 and R57 are independently hydrogen, alkyl, or alkenyl; or

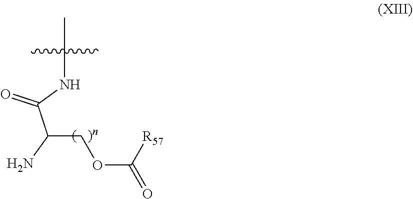

where n=0 to 5, and

R57 is hydrogen, alkyl, alkenyl, aryl, heteroaryl, cyclo, or heterocyclo;

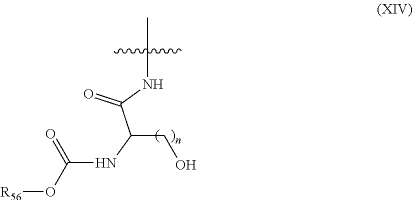

where n=0 to 5, and

R56 of Formula (XIV) is hydrogen, aryl, heteroaryl, alkyl, ester, or alkenyl.

In yet another embodiment, R5 comprises a formula according to Formula (XV):

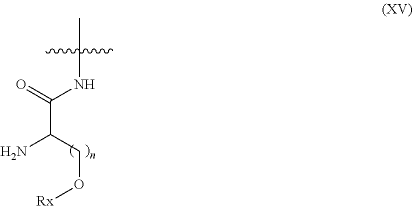

where Rx=phosphate or phosphonate and n=0 to 5.

In an embodiment, when Rx=phosphate, the phosphate comprises one of:

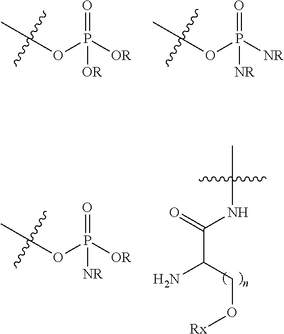

wherein R=hydrogen, alkyl, or alkenyl and n=0 to 5.

In particular embodiments, R5 is selected from a member from the group consisting of:

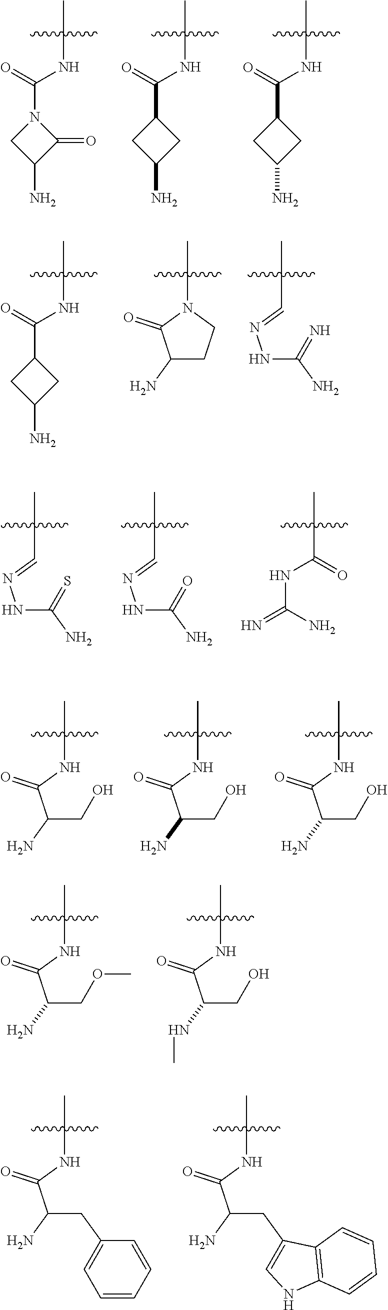

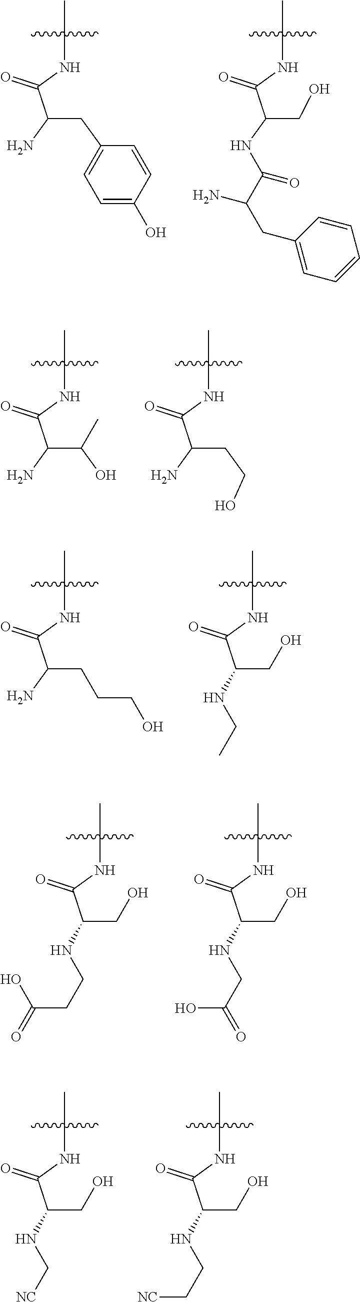

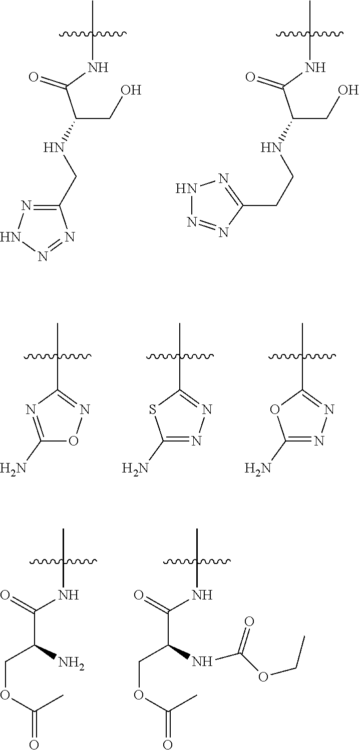

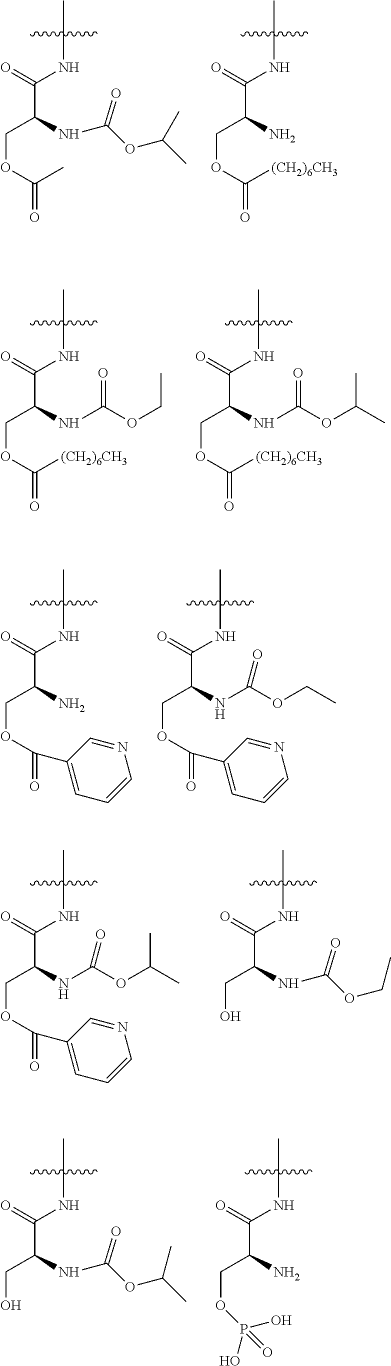

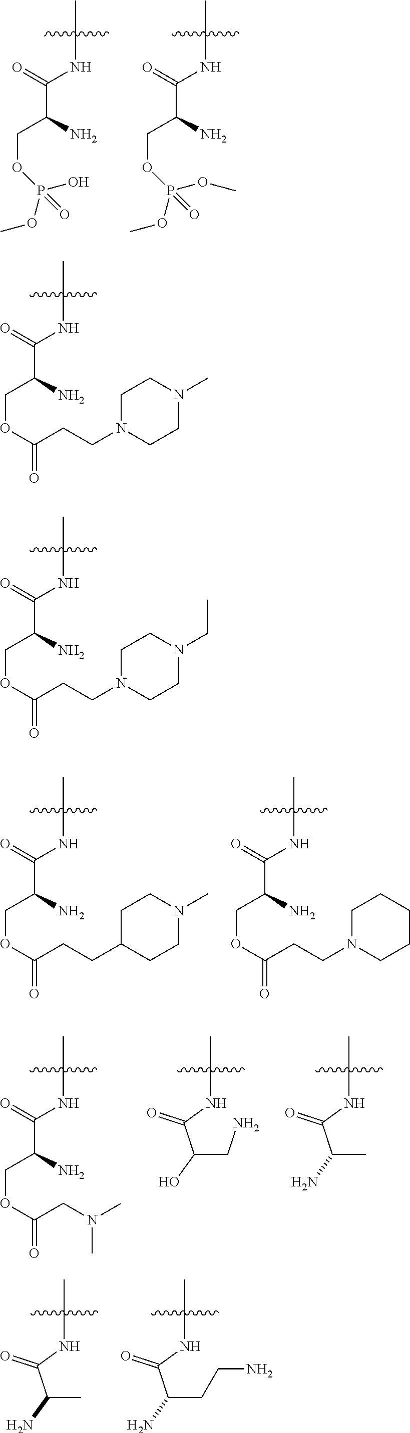

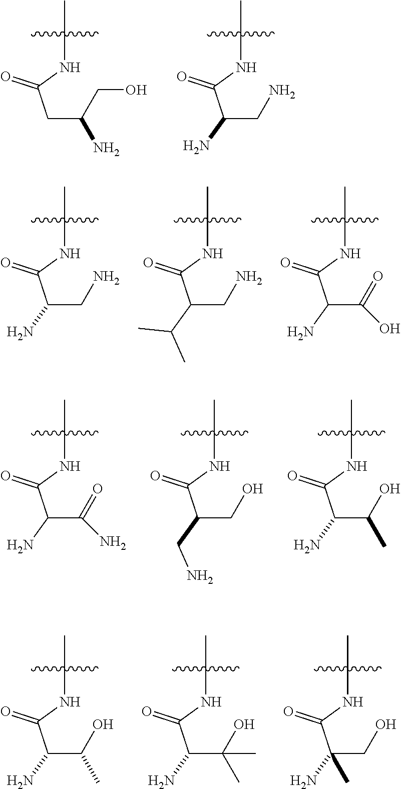

### Definitions

In the present application, the following terms are defined. Any undefined terms shall have their art recognized meanings.

As used herein, H refers to hydrogen.

As used herein, D refers to deuterium.

As used herein, the term “lower” along with a compound class name (e.g., alkyl, aryl) refers to such groups of the type specified with 8 or fewer total carbon atoms, and in certain embodiments, 4 or fewer total carbon atoms.

As used herein, the term “alkyl” provided alone or as part of a larger moiety. Thus, the term alkyl includes substituted or unsubstituted saturated straight-chain, cyclic, or branched aliphatic groups, as well as substituted alkyl groups, including but not limited to alkoxy, haloalkyl, arylalkyl, alkylamine, cycloalkyl, dialkyamine, alkylamino, dialkyamino alkylcarbonyl, alkoxycarbonyl, alkylamino, alkylcarboxylic acid, alkylcarboxylate, alkyl nitrile, and alkylheteroaryl, and the like.

As used herein, the term “lower alkyl” thus refers to an alkyl group having 8 or fewer carbon atoms, and in certain embodiments 4 or fewer carbon atoms, and may include any of lower alkoxy, lower haloalkyl, lower arylalkyl, lower alkylamine, lower cycloalkyl, lower cycloalkylalkyl, lower dialkyamine, lower alkylamino, lower dialkyamino, lower alkylcarbonyl, lower alkoxycarbonyl include straight and branched saturated chains comprising one to eight carbon atoms. In an embodiment, the lower alkyl comprises a lower cycloalkyl. In other embodiments, the lower alkyl comprises methyl, ethyl, propyl, or butyl.

As used herein, the term “alkoxy” comprises a group —OR, wherein R, for example, is a straight or branched chain alkyl group as defined above. In an embodiment, alkoxy is thus understood to include haloalkyl.

As used herein, the term “aryl” refers to an unsaturated cyclic moiety comprising at least one aromatic ring.

As used herein, the term “cycloalkyl” refers to a saturated carbocyclic ring which includes a plurality of carbon atoms, such from three to about twelve carbons (“C3-C12”). Exemplary cycloalkyl groups include cyclopropyl, cyclobutyl, cyclopentyl and cyclohexyl.

As used herein, the term “carbocyclic” refers to a saturated or unsaturated cyclic ring of carbon atoms.

As used herein, the term “ether” refers to an organic compound containing the functional group RO—R′, where R and R′ may comprise an organic or inorganic functional group.

As used herein, the term “halogen” refers to fluoro, bromo, chloro and iodo substituents.

As used herein, the term “heteroaryl” refers to an aromatic ring system with one or more rings in which at least one member of a ring is a heteroatom other than carbon, such as N, O, or S.

As used herein, the term “heterocyclic” or “heterocyclo” refers to any ring system containing carbon and at least one element other than carbon, such as N, O, or S.

As used herein, the term “lower cycloalkyl” refers to a cycloalkyl having fewer than 6 carbon atoms, and in certain embodiment fewer than 4 carbon atoms. In a particular embodiment, the cycloalkyl comprises cyclopropyl.

As used herein, the term “O-lower alkyl” (or alkoxy group) refers to linear, branched, or cyclic oxygen-containing alkyl groups, the alkyl portions having eight or fewer carbon atoms. Exemplary O-lower alkoxy groups include methoxy, ethoxy, propoxy, butoxy and tert-butoxy.

As used herein, p-fluoro refers to

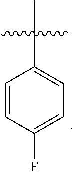 [SMILES: *c1ccc(F)cc1]

As used herein, the term “stereoisomer” refers to all isomers of individual molecules that differ only in the orientation of their atoms in space.

### Further Specific Embodiments

In accordance with certain embodiments, the compounds comprise compounds of Formula (XVI), wherein:

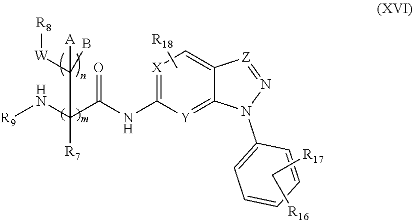

R7=H or methyl

R8=H or COalkyl

R9=H

R16=H or methyl

R17=H or methyl

R18=H or methyl

W=O, N, or methyl

X=CR18, or N

Y=CH

Z=CR18, or N

A=H or CH₃

B=H or CH₃

n=1 or 2

m=1 or 2

In particular embodiments, X=N to provide embodiments comprising 5-aza compounds.

In accordance with another aspect, the compounds comprise compounds of Formula (I), wherein:

A, B, R7, R8, R16, R17, R18=H

R11=CH₃

X, Y, Z=CH

W=O or NH

n=1 or 2

m=1 or 2

In accordance with another aspect, the compounds described herein comprise a member selected from Table 1 below. Table 1 provides a list of exemplary species of compounds developed in accordance with an aspect of the present invention. It is understood that the present invention is not so limited to the compounds listed in Table 1. In addition, Table 1 includes the IUPAC name for each compound as well as an observed mass. Further, Table 1 shows various IC₅₀ values as determined using the coupled enzyme assay for KHK activity described in the Examples below.

TABLE 1

| | | | Nominal | KHK, |
|---|---|---|---|---|
| | | | MS | IC₅₀ |
| # | Structure | Name | (Obs) | (μm) |
| 1 | 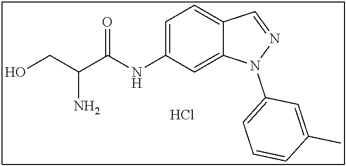 [SMILES: Cc1cccc(-n2ncc3ccc(NC(=O)C(N)CO)cc32)c1 ; Cl] | 2-amino-3-hydroxy-N- (1-(m-tolyl)-1H-indazol- 6-yl)propanamide hydrochloride | 311.0 | 0.12 |
| 2 | 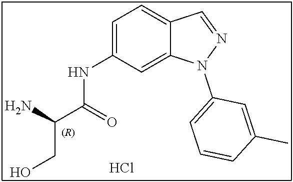 | (R)-2-amino-3- hydroxy-N-(1-(m-tolyl)- 1H-indazol-6- yl)propanamide hydrochloride | 311.0 | 0.12 |
| 3 | 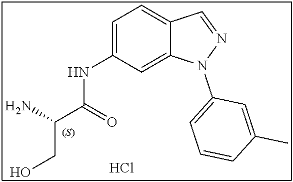 | (S)-2-amino-3- hydroxy-N-(1-(m-tolyl)- 1H-indazol-6- yl)propanamide hydrochloride | 311.0 | 0.04 |
| 4 | 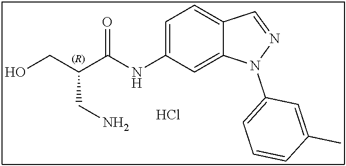 | (R)-3-amino-2- (hydroxymethyl)-N-(1- (m-tolyl)-1H-indazol-6- yl)propanamide hydrochloride | 325.0 | 0.62 |
| 5 | 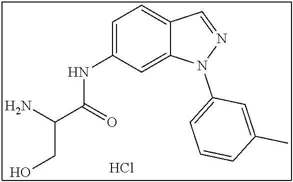 [SMILES: Cc1cccc(-n2ncc3ccc(NC(=O)C(N)CO)cc32)c1 ; Cl] | 3-amino-2-hydroxy-N- (1-(m-tolyl)-1H-indazol- 6-yl)propanamide hydrochloride | 311.0 | 0.52 |
| 6 | 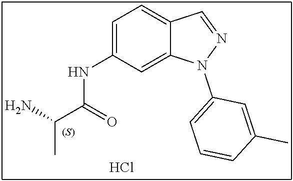 | (S)-2-amino-N-(1-(m- tolyl)-1H-indazol-6- yl)propanamide hydrochloride | 295.0 | 0.11 |
| 7 | 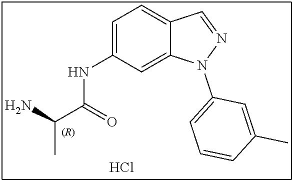 | (R)-2-amino-N-(1-(m- tolyl)-1H-indazol-6- yl)propanamide hydrochloride | 295.0 | 0.27 |
| 8 | 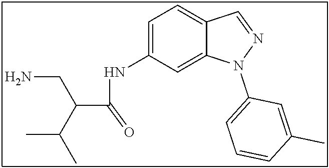 [SMILES: Cc1cccc(-n2ncc3ccc(NC(=O)C(CN)C(C)C)cc32)c1] | 2-(aminomethyl)-3- methyl-N-(1-(m-tolyl)- 1H-indazol-6- yl)butanamide hydrochloride | 337.1 | 4.9 |
| 9 | 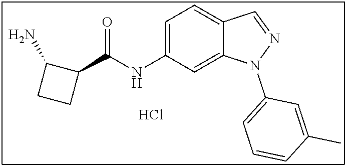 [SMILES: Cc1cccc(-n2ncc3ccc(NC(=O)[C@H]4CC[C@@H]4N)cc32)c1 ; Cl] | trans-2-amino-N-(1-(m- tolyl)-1H-indazol-6-yl) cyclobutanecarboxamide hydrochloride | 321.1 | 0.67 |
| 10 | 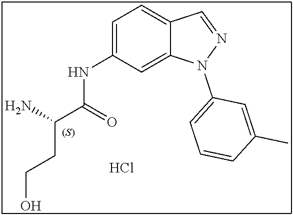 | (S)-2-amino-4- hydroxy-N-(1-(m-tolyl)- 1H-indazol-6- yl)butanamide hydrochloride | 325.1 | 0.4 |
| 11 | 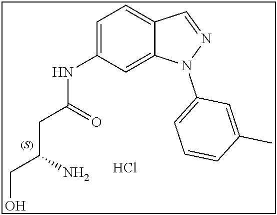 | (S)-3-amino-4- hydroxy-N-(1-(m-tolyl)- 1H-indazol-6- yl)butanamide hydrochloride | 325.1 | 1.2 |
| 12 | 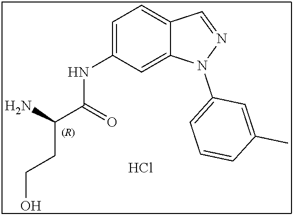 | (R)-2-amino-4- hydroxy-N-(1-(m-tolyl)- 1H-indazol-6- yl)butanamide hydrochloride | 325.1 | 0.86 |
| 13 | 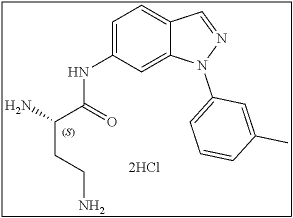 | (S)-2,4-diamino-N-(1- (m-tolyl)-1H-indazol-6- yl)butanamide | 324.1 | 0.35 |
| 14 | 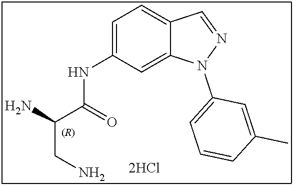 | (R)-2,3-diamino-N-(1- (m-tolyl)-1H-indazol-6- yl)propanamide dihydrochloride | 310.0 | 1.1 |
| 15 | 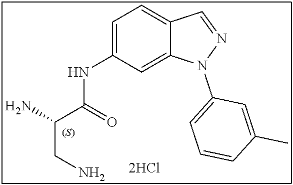 | (S)-2,3-diamino-N-(1- (m-tolyl)-1H-indazol-6- yl)propanamide dihydrochloride | 310.0 | 0.63 |
| 16 | 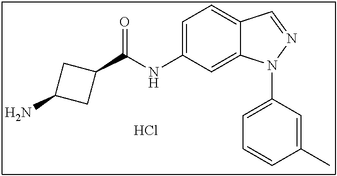 [SMILES: Cc1cccc(-n2ncc3ccc(NC(=O)[C@H]4C[C@@H](N)C4)cc32)c1 ; Cl] | (cis)-3-amino-N-(1-(m- tolyl)-1H-indazol-6-yl) cyclobutanecarboxamide hydrochloride | 321.0 | 0.6 |
| 17 | 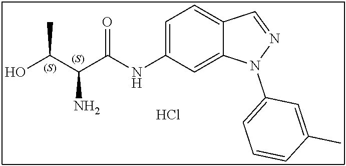 | (2S,3S)-2-amino-3- hydroxy-N-(1-(m-tolyl)- 1H-indazol-6- yl)butanamide hydrochloride | 325.0 | 7 |
| 18 | 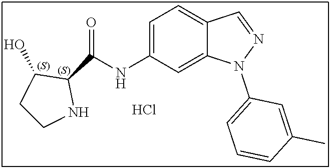 | (2S,3S)-3-hydroxy-N- (1-(m-tolyl)-1H-indazol- 6-yl)pyrrolidine-2- carboxamide hydrochloride | 336.9 | 0.7 |
| 19 | 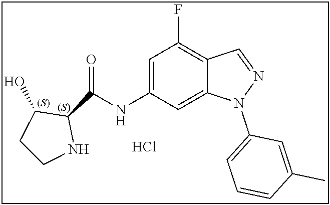 | (2S,3S)-N-(4-fluoro-1- (m-tolyl)-1H-indazol-6- yl)-3- hydroxypyrrolidine-2- carboxamide hydrochloride | 354.9 | 0.52 |
| 20 | 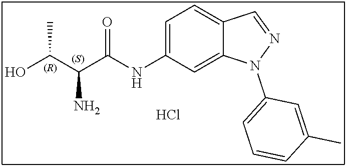 | (2S,3R)-2-amino-3- hydroxy-N-(1-(m-tolyl)- 1H-indazol-6- yl)butanamide hydrochloride | 325.0 | 0.72 |
| 21 | 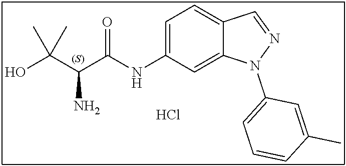 | (S)-2-amino-3- hydroxy-3-methyl-N-(1- (m-tolyl)-1H-indazol-6- yl]butanamide hydrochloride | 338.9 | 3.6 |
| 22 | 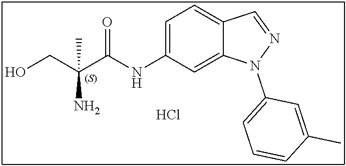 | (S)-2-amino-3- hydroxy-2-methyl-N-(1- (m-tolyl)-1H-indazol-6- yl)propanamide hydrochloride | 325.0 | 0.42 |
| 23 | 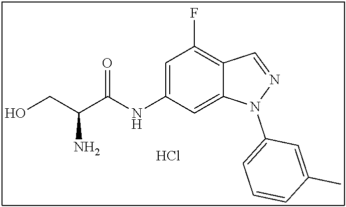 [SMILES: Cc1cccc(-n2ncc3c(F)cc(NC(=O)[C@@H](N)CO)cc32)c1 ; Cl] | (S)-2-amino-N-(4- fluoro-1-(m-tolyl)-1H- indazol-6-yl)-3- hydroxypropanamide hydrochloride | 329.1 | 0.024 |
| 24 | 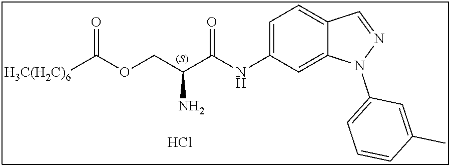 | (S)-2-amino-3-oxo-3- ((1-(m-tolyl)-1H- indazol-6- yl)amino)propyl octanoate hydrochloride | 437.0 | 12 |
| 25 | 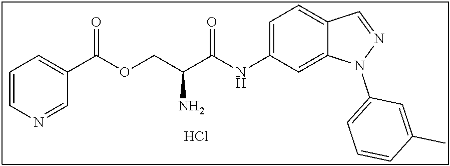 [SMILES: Cc1cccc(-n2ncc3ccc(NC(=O)[C@@H](N)COC(=O)c4cccnc4)cc32)c1 ; Cl] | (S)-2-amino-3-oxo-3- ((1-(m-tolyl)-1H- indazol-6- yl)amino)propyl nicotinate hydrochloride | 415.9 | >1000 |
| 26 | 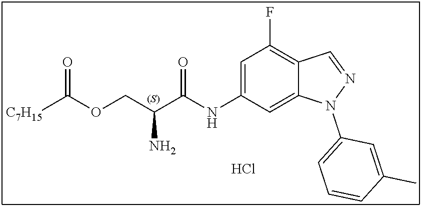 | (S)-2-amino-3-((4- fluoro-1-(m-tolyl)-1H- indazol-6-yl)amino)-3- oxopropyl octanoate hydrochloride | 455.0 | 8 |
| 27 | 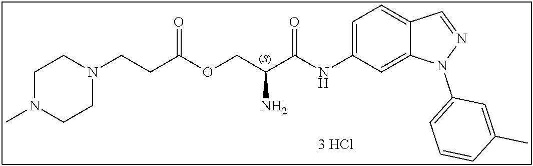 | (S)-2-amino-3-oxo-3- ((1-(m-tolyl)-1H- indazol-6- yl)amino)propyl 3-(4- methylpiperazin-1- yl)propanoate trihydrochloride | 464.3 | N/A |
| 28 | 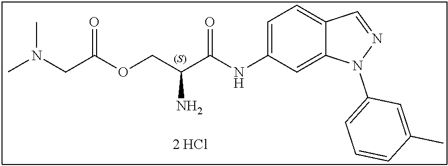 | (S)-2-amino-3-oxo-3- ((1-(m-tolyl)-1H- indazol-6- yl)amino)propyl dimethylglycinate dihydrochloride | 395.2 | N/A |
| 29 | 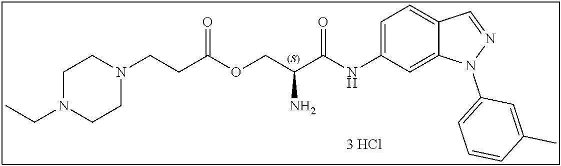 | (S)-2-amino-3-oxo-3- ((1-(m-tolyl)-1H- indazol-6- yl)amino)propyl 3-(4- ethylpiperazin-1- yljpropanoate trihydrochloride | 478.3 | N/A |
| 30 | 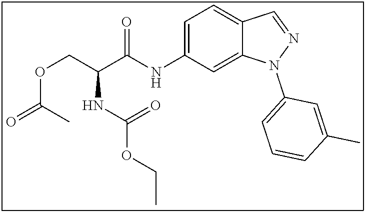 [SMILES: CCOC(=O)N[C@@H](COC(C)=O)C(=O)Nc1ccc2cnn(-c3cccc(C)c3)c2c1] | (S)-2- ((ethyloxycarbonyl) amino)-3-oxo-3-((1-(m- tolyl)-1H-indazol-6- yl)amino)propyl acetate | 424.9 | 24 |
| 31 | 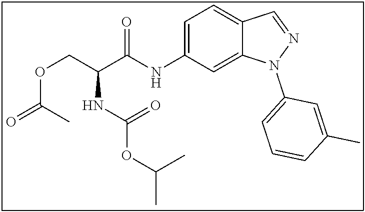 [SMILES: CC(=O)OC[C@H](NC(=O)OC(C)C)C(=O)Nc1ccc2cnn(-c3cccc(C)c3)c2c1] | (S)-2- ((isopropoxycarbonyl) amino)-3-oxo-3-((1-(m- tolyl)-1H-indazol-6- yl)amino)propyl acetate | 438.9 | 30 |
| 32 | 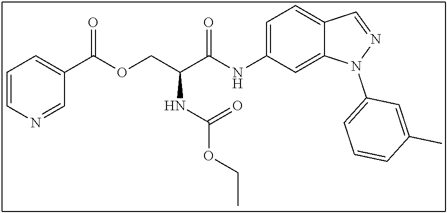 [SMILES: CCOC(=O)N[C@@H](COC(=O)c1cccnc1)C(=O)Nc1ccc2cnn(-c3cccc(C)c3)c2c1] | (S)-2- ((ethyloxycarbonyl) amino)-3-oxo-3-((1-(m- tolyl)-1H-indazol-6- yl)amino)propyl nicotinate | 487.9 | >1000 |
| 33 | 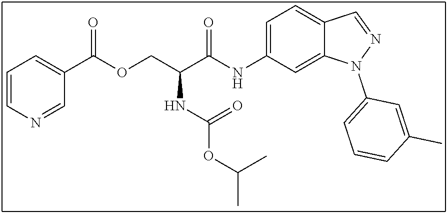 [SMILES: Cc1cccc(-n2ncc3ccc(NC(=O)[C@H](COC(=O)c4cccnc4)NC(=O)OC(C)C)cc32)c1] | (S)-2- ((isopropoxycarbonyl) amino)-3-oxo-3-((1-(m- tolyl)-1H-indazol-6- yl)amino)propyl nicotinate | 501.9 | >1000 |
| 34 | 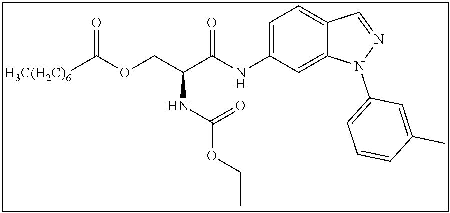 | (S)-2- ((ethoxycarbonyl) amino)-3-oxo-3-((1-(m- tolyl)-1H-indazol-6- yl)amino)propyl octanoate | 510.0 | >1000 |
| 35 | 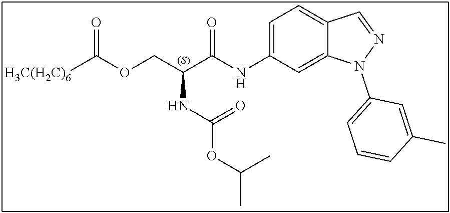 | (S)-2- ((isoproxycarbonyl) amino)-3-oxo-3-((1-(m- tolyl)-1H-indazol-6- yl)amino)propyl octanoate | 524.1 | 200 |
| 36 | 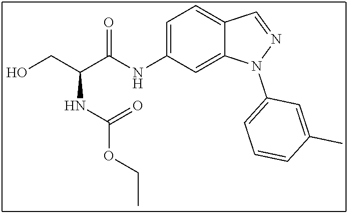 [SMILES: CCOC(=O)N[C@@H](CO)C(=O)Nc1ccc2cnn(-c3cccc(C)c3)c2c1] | (S)-ethyl (3-hydroxy-1- oxo-1-((1-(m-tolyl)-1H indazol-6- yl)amino)propan-2- yl)carbamate | 383.0 | 0.4 |
| 37 | 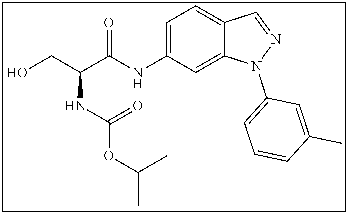 [SMILES: Cc1cccc(-n2ncc3ccc(NC(=O)[C@H](CO)NC(=O)OC(C)C)cc32)c1] | (S)-isopropyl (3- hydroxy-1-oxo-1-((1- (m-tolyl)-1H-indazol-6- yl)amino)propan-2- yl)carbamate | 397.0 | 4 |
| 38 | 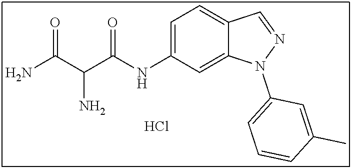 [SMILES: Cc1cccc(-n2ncc3ccc(NC(=O)C(N)C(N)=O)cc32)c1 ; Cl] | 2-amino-N1-(1-(m- tolyl)-1H-indazol-6- yl)malonamide hydrochloride | 324.0 | 1.4 |
| 39 | 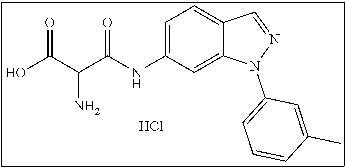 [SMILES: Cc1cccc(-n2ncc3ccc(NC(=O)C(N)C(=O)O)cc32)c1 ; Cl] | 2-amino-3-oxo-3-((1- (m-tolyl)-1H-indazol-6- yl)amino)propanoic acid hydrochloride | 324.1 | 0.7 |
| 40 | 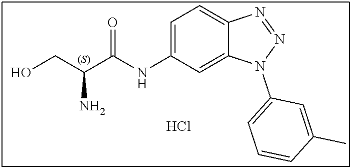 | (S)-2-amino-3- hydroxy-N-(1-(m-tolyl)- 1H- benzo[d][1,2,3]triazol- 6-yl)propanamide hydrochloride | 312.0 | 0.14 |
| 41 | 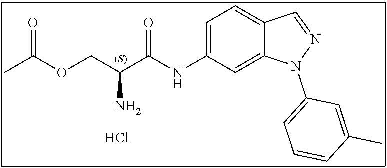 | (S)-2-amino-3-oxo-3- ((1-(m-tolyl)-1H- indazol-6- yl)amino)propyl acetate hydrochloride | 353.0 | 2.7 |
| 42 | 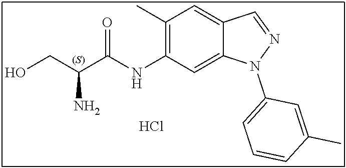 | (S)-2-amino-3- hydroxy-N-(5-methyl-1- (m-tolyl)-1H-indazol-6- yl)propanamide hydrochloride | 335.0 | 1.8 |
| 43 | 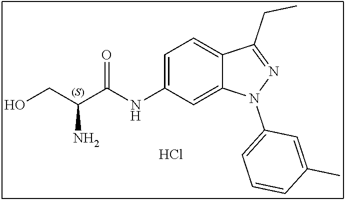 | (S)-2-amino-N-(3- ethyl-1-(m-tolyl)-1H- indazol-6-yl)-3- hydroxypropanamide hydrochloride | 339.0 | 1.86 |
| 44 | 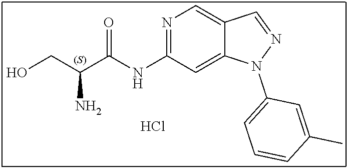 | (S)-2-amino-3- hydroxy-N-(1-(m-tolyl)- 1H-pyrazolo[4,3- c]pyridin-6- yl)propanamide hydrochloride | 312.0 | 15 |
| 45 | 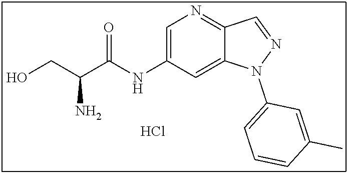 [SMILES: Cc1cccc(-n2ncc3ncc(NC(=O)[C@@H](N)CO)cc32)c1 ; Cl] | (S)-2-amino-3- hydroxy-N-(1-(m-tolyl)- 1H-pyrazolo[4,3- b]pyridin-6- yl)propanamide hydrochloride | 312.0 | 0.25 |
| 46 | 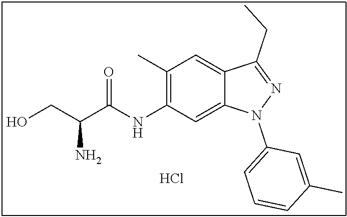 [SMILES: CCc1nn(-c2cccc(C)c2)c2cc(NC(=O)[C@@H](N)CO)c(C)cc12 ; Cl] | (S)-2-amino-N-(3- ethyl-5-methyl-1-(m- tolyl)-1H-indazol-6-yl)- 3-hydroxypropanamide hydrochloride | 353.1 | 1.9 |
| 47 | 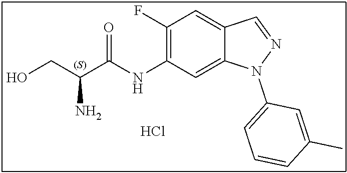 | (S)-2-amino-N-(5- fluoro-1-(m-tolyl)-1H- indazol-6-yl)-3- hydroxypropanamide hydrochloride | 329.0 | N/A |
| 48 | 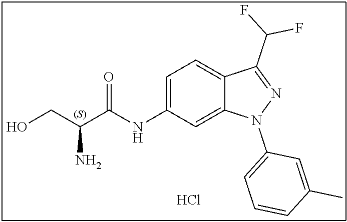 | (S)-2-amino-N-(3- (difluoromethyl)-1-(m- tolyl)-1H-indazol-6-yl)- 3-hydroxypropanamide hydrochloride | 361.0 | 0.46 |

In a particular embodiment, the indazole-based compound comprises a compound according to Formula (XVII) below (also defined as compound 1 herein):

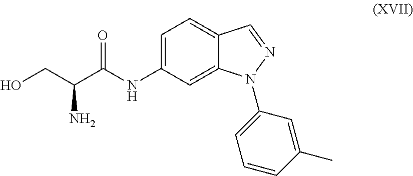 [SMILES: Cc1cccc(-n2ncc3ccc(NC(=O)[C@@H](N)CO)cc32)c1]

In certain embodiments, the compounds disclosed herein comprise fructokinase inhibitors that may be administered to treat or prevent metabolic disorders and diseases, such as those affected mediated to at least an extent by fructose metabolism. Furthermore, it is appreciated that some of the crystalline forms for the compounds according to various embodiments may exist as polymorphs, and as such are intended to be included in the within the scope of this disclosure. In addition, some of the compounds according to various embodiments may form solvates with water (i.e., hydrates) or common organic solvents, and such solvates are also intended to be encompassed within the scope of this disclosure.

In certain embodiments, the compounds described herein may be utilized in the prevention or treatment of a disorder or disease mediated by fructokinase (also referred to as ketohexokinase or KHK). In the disclosure of such methods, the following definitions apply.

The term “prevent” or “preventions” as used herein means either: 1) a reduction in frequency or severity of symptoms commonly associated with a disease or disorder, or 2) a delay or avoidance of additional symptoms associated with the disease or disorder, complete prevention of the disease. One skilled in the art will recognize that wherein various embodiments are directed to methods of prevention or treatment, a subject in need thereof (e.g., a subject in need of prevention) shall include any subject or patient (e.g., a mammal, and in an embodiment a human) who has experienced or exhibited at least one symptom of the disorder, disease or condition to be prevented.

Further, a subject in need thereof may additionally be a subject (e.g., a mammal, and in an embodiment a human) who has not exhibited any symptoms of the disorder, disease, or condition to be prevented, but who has been deemed by a physician, clinician or other medical profession to be at risk of developing said disorder, disease or condition. For example, the subject may be deemed at risk of developing a disorder, disease, or condition (and therefore in need of prevention or preventive treatment) as a consequence of the subject's medical history, including, but not limited to, family history, pre-disposition, co-existing (comorbid) disorders or conditions, genetic testing, and the like.

The term “treat” or “treatment” as used herein means administering a compound to manage the symptoms or underlying cause of a condition with the goal of reducing symptoms or signs of the fructokinase-mediated disorder or disease and either to prevent or to slow progression, to arrest or potentially to reverse manifestations of the disease or disorder, or to inhibit the underlying mechanism(s) causing the disease or disorder.

As used herein, the term “disorder” refers to an illness, a sickness or disease manifested by an interruption, cessation, derangement or abnormality of body functions, systems, or organs.

By “disease,” it is meant any physical condition that damages or interferes with the normal function of a cell, tissue, or organ. In the present invention, the disease comprises one directly or indirectly mediated by fructokinase.

“Exemplary Therapeutic Agents” or therapeutic agent or agents as used herein refers to any compound according to Formulas I and the embodiments encompassed therewith, including but not limited to those featured in Table and by the definitions provided herein. Exemplary Therapeutic Agents include pharmaceutical salts or prodrugs of any compounds according to Formula I.

“Ketohexokinase mediated disease(s) and/or disorder(s)” include, but are not limited to, obesity or elevated abdominal circumference, elevated glucose levels, glucose intolerance, impaired fasting glucose levels (serum glucose 100-125 mg/dl), insulin resistance (as noted by elevated fasting plasma insulin levels or elevated HOMA index), Type I diabetes mellitus, Type II diabetes mellitus, Metabolic Syndrome, lipid disorders characterized by elevated LDL, low HDL, or hypertriglyceridemia, and hypertension (defined as >130/85 mm Hg). In certain embodiments, the ketohexokinase mediated disorder is selected from the group consisting of obesity, Type II diabetes mellitus, and Metabolic Syndrome. Additionally, blocking fructose metabolism via the disclosed fructokinase inhibitors can benefit the following, and may be considered ketohexokinase mediated disease(s) and/or disorder(s) as disclosed herein: rare orphan disease of Hereditary Fructose Intolerance, liver disease (including alcohol-related liver disease (ALD) and non-alcoholic fatty liver disease (NAFLD)), fatty liver, hypertension, cardiac injury from ischemia, certain cancers or metastatic disease (including colon, breast, lung (including adenocarcinoma), acute myelogenous leukemia, hepatocellular, pancreatic, liver cancer or metastases, or gliomas), acute kidney injury from ischemia, heat stress, rhabdomyolysis or radiocontrast, chronic diabetic and nondiabetic renal disease, Alzheimer's disease, alcohol addiction, and attention deficit hyperactivity disorder.

Additional exemplary disorders treatable by the therapeutic agents described herein include fatty liver disease (both nonalcoholic and alcoholic), as well as more progressive forms of nonalcoholic fatty liver disease (including steatohepatitis and cirrhosis). Other exemplary disorders include acute kidney disease due to ischemia, contrast, diabetes, or heat stress, as well as both diabetic and nondiabetic chronic kidney disease.^{5, 8, 9} Ischemia to other organs, including the heart, are also mediated by fructokinase and may be associated with benefit by inhibitors according to various embodiments.¹⁰

Hereditary fructose intolerance is a rare orphan disease that is also amenable to fructokinase therapy. HFI can be associated with hypoglycemia, seizures, lactic acidosis with acute fructose ingestion, and with chronic liver and kidney disease later in life. These conditions can be prevented or treated with fructokinase inhibitor(s)⁷.

Other conditions potentially amenable to fructokinase inhibition therapy for both prevention and/or treatment could include gastrointestinal disorders (celiac disease and Crohn's disease), food-induced allergies (including anaphylaxis), neurological disorders (mania, Alzheimer's and attention deficit disorder), gout, or hyperuricemia.

Fructokinase inhibition may also benefit subjects with obesity, either by helping prevent weight gain, or as a way to prevent rebound of weight following dieting. Inhibitors for fructokinase may also help prevent craving to sugar, HFCS or other compounds that contain fructose. Fructokinase inhibitors may also block weight gain from foods that do not contain fructose (such as nonfructose containing carbohydrates or salt) as they inhibit endogenous fructose that is generated in response to eating these foods.

The terms “subject,” “individual,” “host,” and “patient,” are used interchangeably herein to refer to an animal being treated with one or more exemplary compounds as taught herein, including, but not limited to, simians, humans, avians, felines, canines, equines, rodents, bovines, porcines, ovines, caprines, mammalian farm animals, mammalian sport animals, and mammalian pets. A suitable subject for various embodiments can be any animal, preferably a human, that is suspected of having, has been diagnosed as having, or is at risk of developing a disease that can be ameliorated, treated or prevented by administration of one or more exemplary agents.

As used herein “therapeutically effective amount” refers to an amount which, when administered in a proper dosing regimen, is sufficient to reduce or ameliorate the severity, duration, or progression of the disease disorder being treated, prevent the advancement of the disease, or disorder being treated, cause the regression of the disease or disorder being treated, or enhance or improve the prophylactic or therapeutic effect(s) of another therapy. The full therapeutic effect does not necessarily occur by administration of one dose and may occur only after administration of a series of doses. Thus, a therapeutically effective amount may be administered in one or more administrations per day for successive days.

As used herein, the terms “administering” or “administration” of an agent, drug, or peptide to a subject includes any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the terms “co-administered, “co-administering,” or “concurrent administration”, when used, for example with respect to administration of an exemplary therapeutic agent with another (one or more) exemplary therapeutic agent, or a conjunctive agent along with administration of an exemplary therapeutic agent refers to administration of the exemplary therapeutic agent and the other exemplary therapeutic agent and/or conjunctive agent such that both can simultaneously achieve a physiological effect. The two or more agents, however, need not be administered together. In certain embodiments, administration of one agent can precede administration of another, however, such co-administering typically results in the agents being simultaneously present in the body (e.g. in the plasma) of the subject.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, any exemplified reaction step(s) may be performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example, wherein two steps of a process recite an organic or inorganic base as a reagent, the organic or inorganic base selected for the first step may be the same or different than the organic or inorganic base of the second step. Further, one skilled in the art will recognize that wherein a reaction step of various embodiments may be carried out in a variety of solvents or solvent systems, said reaction step may also be carried out in a mixture of the suitable solvents or solvent systems.

Examples of suitable solvents, bases, reaction temperatures, and other reaction parameters and components are provided in the detailed description which follows herein. One skilled in the art will recognize that the listing of specific examples is not intended, and should not be construed, as limiting in any way the various embodiments set forth in the claims which follow thereafter.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

For use in medicine, the salts of the compounds of various embodiments refer to non-toxic “pharmaceutically acceptable salts.” Other salts may, however, be useful in the preparation of compounds according to various embodiments or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid, or phosphoric acid. Furthermore, where the compounds of various embodiments carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1 S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid, and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine, and zinc hydroxide.

Various embodiments may include prodrugs of the compounds disclosed herein. As used herein the term “prodrug” refers to a biologically inactive compound that can be metabolized in the body to produce a drug. In general, such prodrugs may be functional derivatives of the compounds which are readily convertible in vivo into the required compound. Thus, in the methods of treatment of various embodiments, the term “administering” shall encompass the treatment of the various disorders described with the compound specifically disclosed or with a compound which may not be specifically disclosed, but which converts to the specified compound, e.g., the compounds of Formula (I) in vivo after administration to the patient. Conventional procedures for the selection and preparation of suitable prodrug derivatives are well-known to those of ordinary skill in the art.

### Pharmaceutical Compositions and Dosing

In certain embodiments, compounds disclosed herein are useful in the treatment of disorders mediated by fructokinase (aka KHK or ketohexokinase). Various embodiments may, therefore, provide a method of treating disorders mediated by ketohexokinase comprising administering to a subject in need thereof a therapeutically effective amount of any of the compounds of formulas I or II as herein defined. According to various embodiments, a compound of formula I or II may be administered in a therapeutically effective amount in the range of from about 0.01 mg/kg of body weight to about 20 mg/kg of body weight, or any amount or range therein. For example, a compound of formula I or II may be administered in a therapeutically effective amount in the range having a lower limit and/or an upper limit. The range can include or exclude the lower limit and/or the upper limit. The lower limit and/or upper limit can be selected from about 0.01, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, and 20 mg/kg of body weight. For example, according to certain embodiments, a compound of formula I or II may be administered in a therapeutically effective amount in the range of from about 1 mg/kg of body weight to about 15 mg/kg of body weight, or any combination of lower limits and upper limits described.

Various embodiments may further comprise pharmaceutical compositions containing one or more compounds of formulas I or II with a pharmaceutically acceptable carrier. Pharmaceutical compositions containing one or more of the compounds according to various embodiments as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus, for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions according to various embodiments, one or more compounds of according to various embodiments as the active ingredient may be intimately admixed with a pharmaceutical carrier according to conventional pharmaceutical compounding techniques, which carrier may take a wide variety of forms depending on the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents, and the like; for solid oral preparations such as, for example, powders, capsules, caplets, gelcaps and tablets, suitable carriers and additives include starches, sugars, diluents, granulating agents, lubricants, binders, disintegrating agents, and the like.

Because of their ease in administration, tablets and capsules represent the most advantageous oral dosage unit form, in which case solid pharmaceutical carriers are obviously employed. If desired, tablets may be sugar-coated or enteric-coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above.

The pharmaceutical compositions according to various embodiments may comprise an active ingredient in an amount, per unit dosage unit, e.g., tablet, capsule, powder, injection, suppository, teaspoonful and the like, of from about 0.01 to about 1500 mg or any amount or range therein. For example, the pharmaceutical compositions according to various embodiments may contain an active ingredient in an amount, per unit dosage unit, e.g., tablet, capsule, powder, injection, suppository, teaspoonful and the like, within a range having a lower limit and/or an upper limit. The range may include or exclude the lower limit and/or the upper limit. The lower limit and/or upper limit can be selected from about 0.01, 0.5, 1, 5, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, and 2000 mg. For example, according to certain embodiments, the pharmaceutical compositions according to various embodiments may contain an active ingredient in an amount, per unit dosage unit, e.g., tablet, capsule, powder, injection, suppository, teaspoonful and the like, of from about 0.01 to about 1500 mg, or any combination of lower limits and upper limits described.

Furthermore, the pharmaceutical compositions according to various embodiments may be administered at a dosage of from about 0.01 to about 100 mg/kg/day, or any amount or range therein, preferably from about 0.01 to about 20 mg/kg/day, or any amount or range therein. The dosages, however, may be varied depending upon the requirement of the patients, the severity of the condition being treated, and the compound being employed. The use of either daily administration or post-periodic dosing may be employed. For example, the pharmaceutical compositions according to various embodiments may be administered at a dosage within a range having a lower limit and/or an upper limit. The range may include or exclude the lower limit and/or the upper limit. The lower limit and/or upper limit can be selected from about 0.01, 0.5, 1, 5, 10, 20, 30, 40, 50, 60, 70, 80, 90, 100, 110, 120, 130, 140, 150, 160, 170, 180, 190, and 200 mg/kg/day. For example, according to certain embodiments, the pharmaceutical compositions according to various embodiments may be administered at a dosage of from about 0.5 to about 50 mg/kg/day, or any combination of lower limits and upper limits described.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound according to various embodiments, or a pharmaceutically acceptable salt thereof.

When referring to these pre-formulation compositions as homogeneous, it is meant that the active ingredient is dispersed evenly throughout the composition so that the composition may be readily subdivided into equally effective dosage forms such as tablets, pills and capsules. This solid pre-formulation composition is then subdivided into unit dosage forms of the type described above containing from about 0.01 to about 1000 mg of the active ingredient of various embodiments, or any amount or range therein. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of various embodiments may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs, and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, or gelatin.

The method of treating fructokinase (KHK) mediated conditions, disorders, or diseases described in various embodiments may also be carried out using a pharmaceutical composition comprising any of the compounds as defined herein and a pharmaceutically acceptable carrier. The pharmaceutical composition may contain between about 0.01 mg and 1000 mg of the compound, or any amount or range therein; preferably about 0.5 to 500 mg of the compound, or any amount or range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds according to various embodiments may be administered in a single daily dose, or the total daily dosage may be administered in divided doses of two, three or four times daily. Furthermore, compounds according to various embodiments can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents, and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride, and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition according to certain embodiments, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Those having ordinary skill in the art will be well-apprised of various pharmaceutically acceptable carriers.

Exemplary therapeutic agents according to various embodiments may be administered in any of the foregoing compositions and according to dosage regimens established in the art whenever treatment of disorders mediated by KHK is required.

The daily dosage of the products may be varied over a wide range from about 0.01 to about 1,500 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.01 mg/kg to about 100 mg/kg of body weight per day, or any amount or range therein. Preferably, the range is from about 0.01 to about 50.0 mg/kg of body weight per day, or any amount or range therein. More preferably, from about 0.01 to about 20.0 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet, and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder. One skilled in the art will further recognize that human clinical trials including first-in-human, dose ranging and efficacy trials, in healthy patients and/or those suffering from a given disorder, may be completed according to methods well known in the clinical and medical arts.

The determination of a therapeutically effective dose of the exemplary therapeutic agents is well within the capability of those skilled in the art. A therapeutically effective dose refers to that amount of active ingredient which modulates KHK activity compared to that which occurs in the absence of the therapeutically effective dose. Therapeutic efficacy and toxicity, e.g., ED50 (the dose therapeutically effective in 50% of the population) and LD50 (the dose lethal to 50% of the population), can be determined by standard pharmaceutical procedures in cell cultures or experimental animals. The dose ratio of toxic to therapeutic effects is the therapeutic index, and it can be expressed as the ratio, LD50/ED50.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

In typical embodiments, therapeutic agents according to various embodiments may reduce the activity of a KHK polypeptide by at least about 10-100 percent. Various embodiments may provide a reduction of KHK activity of at least 50, 75, 90, or 100% relative to the absence of the exemplary therapeutic agent. For example, various embodiments may provide a reduction of KHK activity within a range having a lower limit and/or an upper limit. The range may include or exclude the lower limit and/or the upper limit. The lower limit and/or upper limit can be selected from about 10, 15, 20, 25, 30, 35, 40, 45, 50, 55, 60, 65, 70, 75, 80, 85, 90, 95, and 100%. For example, according to certain embodiments, various embodiments may provide a reduction of KHK activity of from about 10 to about 100%, or any combination of lower limits and upper limits described.

### Conjunctive Therapeutic Agents

In any of the composition or method embodiments described herein, any of the exemplary therapeutic agents comprising one or more of the compounds set forth herein can be co-administered with other appropriate agents (conjunctive agent or conjunctive therapeutic agent) for the treatment or prevention of a target disease. Selection of the appropriate conjunctive agents for use in combination therapy can be made by one of ordinary skill in the art, according to conventional pharmaceutical principles. The combination of therapeutic agents can act synergistically or additively to affect the treatment or prevention of the various diseases or disorders described herein. Using this approach, one may be able to achieve therapeutic efficacy with lower dosages of each agent, thus reducing the potential for adverse side effects. Any of the therapeutic methods and compositions comprising an exemplary therapeutic agent described herein can be co-administered with another conjunctive agent to a subject in need of such therapy.

Exemplary conjunctive agents that may be formulated and/or administered with any form of an exemplary therapeutic agent as described herein include, but are not limited to, angiotensin-converting enzyme (ACE) inhibitors, aldosterone antagonists, amphetamines, amphetamine-like agents, Angiotensin II receptor antagonists, anti-oxidants, aldose reductase inhibitors, biguanides, sorbitol dehydrogenase inhibitors, thiazolidinediones (glitazones), thiazide and thiazide-like diuretics, triglyceride synthesis inhibitors, the glifozins, the vaptans (antagonists of vasopressin receptors, including V2 V1a, V1 b and combinations of these), inhibitors of adenosine monophosphate deaminase, uric acid lowering agents, e.g., xanthine oxidase inhibitors, and combinations thereof.

Exemplary ACE inhibitors include, but are not limited to, Benazepril (Lotensin), Captopril, Enalapril (Vasotec), Fosinopril, Lisinopril (Prinivil, Zestril), Moexipril (Univasc), Perindopril (Aceon), Quinapril (Accupril), Ramipril (Altace), Trandolapril (Mavik), and combinations thereof.

Exemplary aldosterone antagonists include, but are not limited to, Spironolactone, Eplerenone, Canrenone (canrenoate potassium), Prorenone (prorenoate potassium), Mexrenone (mexrenoate potassium), and combinations thereof.

Exemplary amphetamines include, but are not limited to, amphetamine, methamphetamine, methylphenidate, p-methoxyamphetamine, methylenedioxyamphetamine, 2,5-dimethoxy-4-methylamphetamine, 2,4,5-trimethoxyamphetamine, and 3,4-methylenedioxymethamphetamine, N-ethylamphetamine, fenethylline, benzphetamine, and chlorphentermine as well as the amphetamine compounds of Adderall.RTM.; actedron; actemin; adipan; akedron; allodene; alpha-methyl-(.+−.)-benzeneethanamine; alpha-methylbenzeneethanamine; alpha-methylphenethylamine; amfetamine; amphate; anorexine; benzebar; benzedrine; benzyl methyl carbinamine; benzolone; beta-amino propylbenzene; beta-phenylisopropylamine; biphetamine; desoxynorephedrine; dietamine; DL-amphetamine; elastonon; fenopromin; finam; isoamyne; isomyn; mecodrin; monophos; mydrial; norephedrane; novydrine; obesin; obesine; obetrol; octedrine; oktedrin; phenamine; phenedrine; phenethylamine, alpha-methyl-; percomon; profamina; profetamine; propisamine; racephen; raphetamine; rhinalator, sympamine; simpatedrin; simpatina; sympatedrine; and weckamine. Exemplary amphetamine-like agents include but are not limited to methylphenidate. Exemplary compounds for the treatment of ADD include, but are not limited to, methylphenidate, dextroamphetamine/amphetamine, dextroamphetamine, and atomoxetine (non-stimulant).

Exemplary Angiotensin II receptor antagonists or angiotensin receptor blockers (ARBs) include, but are not limited to losartan, irbesartan, olmesartan, candesartan, valsartan, and combinations thereof.

Exemplary anti-oxidant compounds include but are not limited to L-ascorbic acid or L-ascorbate (vitamin C), menaquinone (vitamin K 2), plastoquinone, phylloquinone (vitamin K 1), retinol (vitamin A), tocopherols (e.g., a, f3, y and o-tocotrienols, ubiquinol, and ubiquione (Coenzyme Q10)); and cyclic or polycyclic compounds including acetophenones, anthroquinones, benzoquiones, biflavonoids, catechol melanins, chromones, condensed tannins, coumarins, curcurmins, flavonoids (catechins and epicatechins), hydrolyzable tannins, hydroxycinnamic acids, hydroxybenzyl compounds, isoflavonoids, lignans, naphthoquinones, neolignans, phenolic acids, phenols (including bisphenols and other sterically hindered phenols, aminophenols and thiobisphenols), phenylacetic acids, phenylpropenes, stilbenes and xanthones. Additional cyclic or polycyclic antioxidant compounds include apigenin, auresin, aureusidin, Biochanin A, capsaicin, catechin, coniferyl alcohol, coniferyl aldehyde, cyanidin, daidzein, daphnetin, deiphinidin, emodin, epicatechin, eriodicytol, esculetin, ferulic acid, formononetin, gernistein, gingerol, 3-hydroxybenzoic acid, 4-hydroxybenzoic acid, 3-hydroxycoumarin, juglone, kaemferol, lunularic acid, luteolin, malvidin, mangiferin, 4-methylumbelliferone, mycertin, naringenin, pelargonidin, peonidin, petunidin, phloretin, p-hydroxyacetophenone, (+)-pinoresinol, procyanidin B-2, quercetin, resveratol, resorcinol, rosmaric acid, salicylic acid, scopolein, sinapic acid, sinapoyl-(S)-maleate, sinapyl aldehyde, syrginyl alcohol, telligrandin umbelliferone and vanillin. Antioxidants may also be obtained from plant extracts, e.g., from blackberries, blueberries, black carrots, chokecherries, cranberries, black currants, elderberries, red grapes and their juice, hibiscus, oregano, purple sweet potato, red wine, rosemary, strawberries, tea (e.g., black, green or white tea), and from various plant ingredients as ellagic acid.

Exemplary aldose reductase inhibitors include, but are not limited to, epalrestat, ranirestat, fidarestat, sorbinil, and combinations thereof.

Exemplary biguanides include, but are not limited to, metformin, and less rarely used phenformin and buformin, proguanil, and combinations thereof.

Exemplary thiazolidinediones include, but are not limited to, troglitazone, pioglitazone, ciglitazone, rosiglitazone, englitazone, and combinations thereof.

Exemplary sorbitol dehydrogenase inhibitors are disclosed in U.S. Pat. Nos. 6,894,047, 6,570,013, 6,294,538, and US Published Patent Application No. 20050020578, the entirety of which are incorporated by reference herein. Exemplary thiazide and thiazide-like diuretics include, but are not limited to, benzothiadiazine derivatives, chlorthalidone, metolazone, and combinations thereof. Exemplary triglyceride synthesis inhibitors include, but are not limited to, diglyceride acyltransferase 1 (DGAT-1) inhibitors. In addition, in certain embodiments, therapeutic agents comprising one or more of the compounds set forth herein may be utilized in combination with glifozins used to treat type 2 diabetes, including but not limited to empaglifozin, dapaglifozin, canaglifozin, and ertuglifozin.

Exemplary vaptans include tolvaptan, conivaptan and nelivaptan.

Exemplary uric acid lowering agents include, but are not limited to, xanthine oxidase inhibitors, such as allopurinol, oxypurinol, tisopurine, febuxostat, Topiroxostat, inositols (e.g., phytic acid and myo-inositol), and combinations thereof. An exemplary AMP Deaminase inhibitor would include compounds such as described by Admyre et al.¹¹

It is appreciated that suitable conjunctive therapeutic agents for use in various embodiments may also comprise any combinations, prodrugs, pharmaceutically acceptable salts, analogs, and derivatives of the above compounds. In one embodiment, the exemplary therapeutic agent may be administered to the subject along with one or more other conjunctive therapeutic agents that are active in acute and chronic kidney disease. Exemplary conjunctive therapeutic agents for this use include but are not limited to angiotensin-converting enzyme (ACE) inhibitors, aldosterone antagonists, Angiotensin II receptor antagonists, anti-oxidants, aldose reductase inhibitors, biguanides, sorbitol dehydrogenase inhibitors, thiazolidinediones (glitazones), xanthine oxidase inhibitors, and/or any other agent used to treat acute or chronic kidney disease.

In another embodiment, the therapeutic agent may be administered along with conjunctive therapeutic agents in the treatment of metabolic syndrome, obesity, sugar addiction, sugar craving, and attention deficit disorder. Exemplary conjuvant therapeutic agents that may be formulated and/or administered with any form of an exemplary therapeutic agent as described herein include, but are not limited to, angiotensin-converting enzyme (ACE) inhibitors, aldosterone antagonists, amphetamines, amphetamine-like agents, Angiotensin II receptor antagonists, anti-oxidants, aldose reductase inhibitors, sorbitol dehydrogenase inhibitors, thiazide and thiazide-like diuretics, triglyceride synthesis inhibitors, and/or any other agent used to treat metabolic syndrome, obesity, sugar addiction, sugar craving, and/or attention deficit disorders.

### Therapeutic Methods

According to other embodiments, one or more exemplary therapeutic agents are administered in a therapeutically effective amount to treat a KHK mediated disorder or disease in a subject in need. A subject in need is one who has exhibited one or more symptoms of any KHK mediated disorder or disease including presence of a testable physiological marker of the disease in a biological sample (such as blood, serum, saliva or urine), who is at risk of developing a KHK mediated disorder or disease, and/or who has been diagnosed by a medical practitioner to be at risk of developing and/or to have a KHK-mediated disease or disorder.

The one or more exemplary therapeutic agents may be administered in a pharmaceutical composition. The pharmaceutical composition may include one or more therapeutic conjunctive agents.

### EXAMPLES

### Synthesis of Exemplary Therapeutic Agents

General schemas for synthesizing a variety of exemplary therapeutic agents are detailed in this section, including schemas for synthesizing the indazole-based compounds described herein. A person having ordinary skill in the art will understand that the present disclosure is not limited by these exemplary schemas and will be able to readily envision variations.

### Preparation of 1-(m-tolyl)-1H-indazol-6-amine hydrochloride

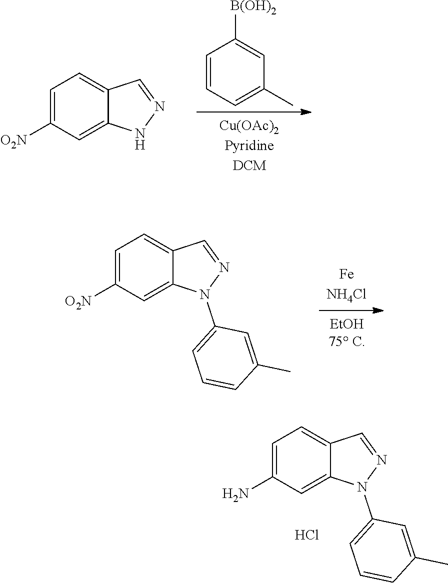

Step 1: In a 3-neck round bottom flask equipped with a mechanical stirrer, 6-nitro-1H-indazole (15.0 g, 92.0 mmol), m-tolylboronic acid (15.0 g, 110.3 mmol), Cu(OAc)₂ (25.0 g, 137.6 mmol), and pyridine (15.6 mL, 151.4 mmol) were combined in DCM (750 mL). The resultant suspension was stirred for 14 h. TLC indicated the complete consumption of the starting indazole. The mixture filtered through a small silica plug to remove copper salts and which was rinsed with additional DCM (150 mL). The filtrate was concentrated to dryness, resuspended in DCM (200 mL), transferred to a separatory funnel and washed with an NH₄Cl solution (3×75 mL). The organic layer was dried over Na₂SO₄, filtered, and concentrated. The resultant crude was purified by flash column chromatography (5% EtOAc:heptane) to give 6-nitro-1-(m-tolyl)-1H-indazole as a yellow solid (8.0 g, 35% yield). The NMR shows a mix of N1 and N2 isomers in an 8:1 ratio. Compound was taken on to the next step without further purification.

Step 2: 6-nitro-1-(m-tolyl)-1H-indazole (8.0 g, 31.6 mmol), Fe powder (5.3 g, 94.9 mmol), and NH₄Cl (8.4 g, 157.0 mmol) were suspended in 5:1 EtOH:H₂O (240 mL). The reaction mixture was stirred and heated to 75° C. for 3 h. LC/MS indicated a complete conversion to the amino compound. The reaction mixture was filtered through a bed of celite. The celite was rinsed with additional EtOH (60 mL). The filtrate was concentrated to remove ethanol and EtOAc (75 mL) was added. The mixture was diluted with H₂O (25 mL) and the layers were separated. The organic layer was dried over Na₂SO₄, filtered, and concentrated to give the desired freebase which was clean by NMR. The oil was dissolved in EtOAc (25 mL) and treated with 2 N ethereal HCl (5 mL). The mixture was stirred for 11 h and then filtered to give 1-(m-tolyl)-1H-indazol-6-amine hydrochloride as a yellow solid (4.8 g, 59% yield). LC/MS and NMR are consistent.

### Preparation of Compound 1

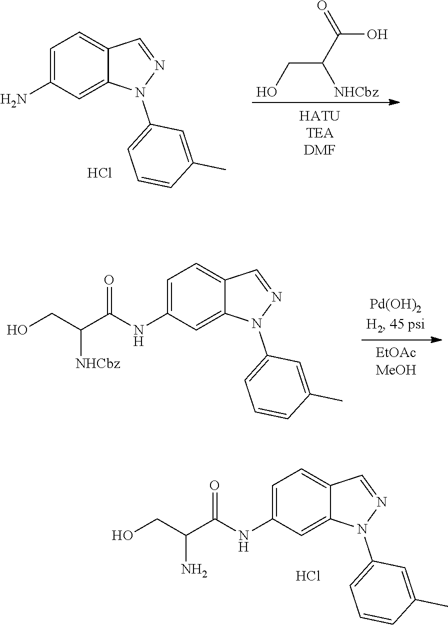 [SMILES: Cc1cccc(-n2ncc3ccc(N)cc32)c1 ; Cl ; O=C(NC(CO)C(=O)O)OCc1ccccc1 ; Cc1cccc(-n2ncc3ccc(NC(=O)C(CO)NC(=O)OCc4ccccc4)cc32)c1 ; Cc1cccc(-n2ncc3ccc(NC(=O)C(N)CO)cc32)c1 ; Cl]

Step 1: To a solution of 1-(m-tolyl)-1H-indazol-6-amine hydrochloride (400 mg, 1.54 mmol) and HATU (760 mg, 2.00 mmol) in DMF (22 mL) at 0° C. was added dropwise a solution of Cbz-DL-Serine (480 mg, 2.01 mmol) and TEA (563 μL, 3.85 mmol) in DMF (8 mL). The reaction mixture was stirred for 3 h, slowly coming to room temperature. LC/MS indicated a complete reaction. The reaction mixture was diluted with a saturated NaHCO₃ solution (40 mL) and extracted into EtOAc (100 mL). The organic layer was rinsed with a brine solution (2×30 mL). The organic layer was then dried over Na₂SO₄, filtered, and concentrated to give a crude oil. The crude was purified by flash column chromatography (50% EtOAc:heptane) to give benzyl (3-hydroxy-1-oxo-1-((1-(m-tolyl)-1H-indazol-6-yl)amino)propan-2-yl)carbamate as an off-white, spongy solid (620 mg, 91% yield). LC/MS and NMR are consistent.

Step 2: Benzyl (3-hydroxy-1-oxo-1-((1-(m-tolyl)-1H-indazol-6-yl)amino)propan-2-yl)carbamate (100 mg, 0.22 mmol) was dissolved in 3:1 EtOAc:MeOH (5 mL) in a hydrogenation flask. The flask was charged with Pd(OH)₂ (25 mg) and the flask was sealed and pressurized with H₂ to 45 psi. The mixture was stirred under pressure for 15 h at which point LC/MS indicated a complete deprotection. The reaction mixture was filtered through a bed of celite and the celite cake was rinsed with additional EtOAc (25 mL). The filtrate was concentrated to give the desired free base. The free base was then dissolved in MTBE (3 mL with a drop of MeOH for solubility) and treated with 2 N ethereal HCl (1 mL). The resultant suspension was filtered to give 2-amino-3-hydroxy-N-(1-(m-tolyl)-1H-indazol-6-yl)propanamide hydrochloride as an off-white solid (40 mg, 51% yield). LC/MS and NMR are consistent.

### Preparation of Compound 2

Compound 2 was prepared in a similar manner to compound 1.

### Preparation of Compound 3

Compound 3 was prepared in a similar manner to compound 1.

### Preparation of Compound 4

Compound 4 was prepared in a similar manner to compound 1.

### Preparation of Compound 5

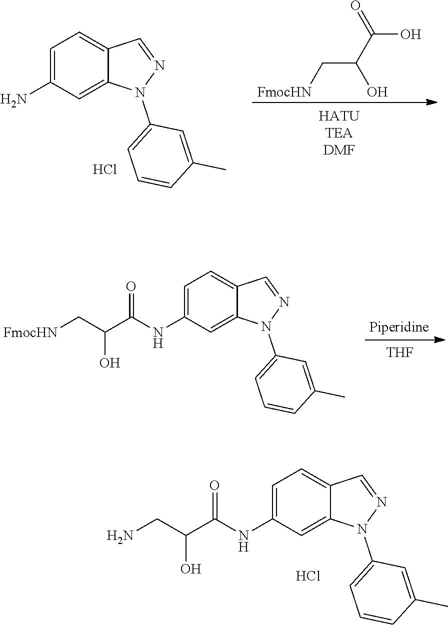

Step 1: To a solution of 1-(m-tolyl)-1H-indazol-6-amine hydrochloride (100 mg, 0.52 mmol), Fmoc-DL-isoserine (166 mg, 0.51 mmol) and HATU (242 mg, 2.00 mmol) in DMF (22 mL) at 0° C. was added DIPEA (222 μL, 1.27 mmol). The reaction mixture was stirred for 15 h, slowly coming to room temperature. LC/MS indicated the desired amide compound was formed. The reaction mixture was diluted with a saturated NaHCO₃ solution (40 mL) and extracted into EtOAc (100 mL). The organic layer was rinsed with a brine solution (2×30 mL). The organic layer was then dried over Na₂SO₄, filtered, and concentrated to give a crude oil. The crude was purified by CombiFlash chromatography (50% EtOAc:heptane) to give (9H-fluoren-9-yl)methyl (2-hydroxy-3-oxo-3-((1-(m-tolyl)-1H-indazol-6-yl)amino)propyl)carbamate as a white solid (110 mg, 49% yield). LC/MS and NMR are consistent.

Step 2: To a solution of (9H-fluoren-9-yl)methyl (2-hydroxy-3-oxo-3-((1-(m-tolyl)-1H-indazol-6-yl)amino)propyl)carbamate (110 mg, 0.21 mmol) in THF (4 mL) was added piperidine (41 μL, 0.42 mmol). The solution was stirred for 16 h at which point LC/MS indicated a complete deprotection. The reaction mixture was concentrated to dryness and the resultant crude material was purified by CombiFlash chromatography (5% MeOH:DCM with 0.1% methanolic ammonia) to yield the desired free base. The free base was dissolved in MTBE (3 mL with a drop of MeOH for solubility) and then treated with 2 N ethereal HCl (1 mL). The resultant suspension was filtered to give 3-amino-2-hydroxy-N-(1-(m-tolyl)-1H-indazol-6-yl)propanamide hydrochloride as a white solid (32 mg, 44% yield). LC/MS and NMR are consistent.

### Preparation of Compound 6

Compound 6 was prepared in a similar manner to compound 5.

### Preparation of Compound 7

Compound 7 was prepared in a similar manner to compound 5.

### Preparation of Compound 8

Compound 8 was prepared in a similar manner to compound 5.

### Preparation of Compound 9

Compound 9 was prepared in a similar manner to compound 5.

### Preparation of Compound 10

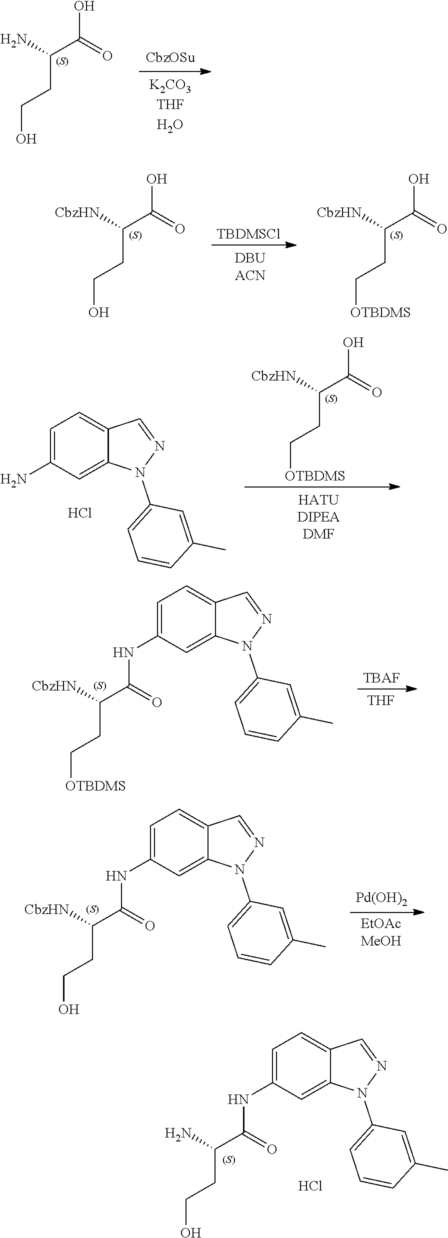

Step 1: To a mixture L-Homoserine (1.0 g, 8.39 mmol) and N-(benzyloxycarbonyloxy)succinimide (2.5 g, 10.03 mmol) in 4:1 THF:H₂O (25 mL) was added K₂CO₃ (1.6 g, 11.58 mmol). The mixture was stirred for 16 h and then the mixture was concentrated to remove THF. The mixture was then diluted with additional H₂O (20 mL) and rinsed with EtOAc (2×20 mL). The organic layers were discarded. The aqueous layer was then acidified with 2 N HCl and then extracted into EtOAc (2×20 mL). This second organic layer was dried over Na₂SO₄, filtered, and concentrated to give (S)-2-(((benzyloxy)carbonyl)amino)-4-hydroxybutanoic acid as an off-white solid (910 mg, 43% yield). LC/MS and NMR are consistent.

Step 2: To a solution of (S)-2-(((benzyloxy)carbonyl)amino)-4-hydroxybutanoic acid (300 mg, 1.18 mmol) in ACN (5 mL) at 0° C. was added DBU (234 μL, 1.57 mmol) followed by TBDMS chloride (213 mg, 1.41 mmol) in 3 portions. The reaction mixture was then stirred for 14 h, slowly coming to room temperature. The mixture was then concentrated, partitioned between EtOAc (20 mL) and H₂O (15 mL). The aqueous layer was back extracted with EtOAc (10 mL). The organic layers were combined, dried over Na₂SO₄, filtered, and concentrated to give (S)-2-(((benzyloxy)carbonyl)amino)-4-((tert-butyldimethylsilyl)oxy)butanoic acid as a colorless syrup (300 mg, 69% yield). LC/MS is consistent.

Step 3: To a solution of 1-(m-tolyl)-1H-indazol-6-amine hydrochloride (211 mg, 0.81 mmol), (S)-2-(((benzyloxy)carbonyl)amino)-4-((tert-butyldimethylsilyl)oxy)butanoic acid (300 mg, 0.82 mmol), and HATU (465 mg, 1.22 mmol) in DMF (5 mL) at 0° C. was added DIPEA (427 μL, 2.45 mmol). The reaction mixture was stirred for 16 h, slowly coming to room temperature. LC/MS indicated only ˜50% conversion to the desired amide. The reaction mixture was diluted with H₂O (20 mL) and extracted into EtOAc (3×10 mL). The combined organic layers were dried over Na₂SO₄, filtered, and concentrated to give a crude oil. The crude was attempted to be purified by CombiFlash chromatography (30% EtOAc:heptane), however the isolated material was a mixture of (S)-benzyl (4-((tert-butyldimethylsilyl)oxy)-1-oxo-1-((1-(m-tolyl)-1H-indazol-6-yl)amino)butan-2-yl)carbamate and the starting 1-(m-tolyl)-1H-indazol-6-amine (350 mg, yield not determined at this step). The material was taken on to the first deprotection step without further purification.

Step 4: To a solution of the crude (S)-benzyl (4-((tert-butyldimethylsilyl)oxy)-1-oxo-1-((1-(m-tolyl)-1H-indazol-6-yl)amino)butan-2-yl)carbamate (350 mg) in THF (5 mL) at 0° C. was added a 1 M solution TBAF in THF (720 μL, 0.72 mmol). The solution was stirred overnight, slowly coming to room temperature. The solution was concentrated and directly purified by CombiFlash chromatography (50% EtOAc:heptane) to give (S)-benzyl (4-hydroxy-1-oxo-1-((1-(m-tolyl)-1H-indazol-6-yl)amino)butan-2-yl)carbamate as an off-white gummy solid (132 mg, 25% yield over two steps). NMR and LC/MS are consistent.

Step 5: (S)-Benzyl (4-hydroxy-1-oxo-1-((1-(m-tolyl)-1H-indazol-6-yl)amino)butan-2-yl)carbamate (128 mg, 0.28 mmol) was dissolved in 1:1 EtOAc:MeOH (5 mL) in a hydrogenation flask. The flask was charged with Pd(OH)₂ (20 mg) and the flask was sealed and pressurized with H₂ to 45 psi. The mixture was stirred under pressure for 15 h at which point LC/MS indicated a complete deprotection. The reaction mixture was filtered through a bed of celite and the celite cake was rinsed with additional EtOAc (25 mL). The filtrate was concentrated to give the desired free base. The free base was then dissolved in MTBE (3 mL, with a drop of MeOH for solubility) and treated with 2 N ethereal HCl (1 mL). The resultant suspension was filtered to give (S)-2-amino-4-hydroxy-N-(1-(m-tolyl)-1H-indazol-6-yl)butanamide hydrochloride as an off-white solid (32 mg, 32% yield). LC/MS and NMR are consistent.

### Preparation of Compound 11

Compound 11 was prepared in a similar manner to compound 10.

### Preparation of Compound 12

Compound 12 was prepared in a similar manner to compound 10.

### Preparation of Compound 13

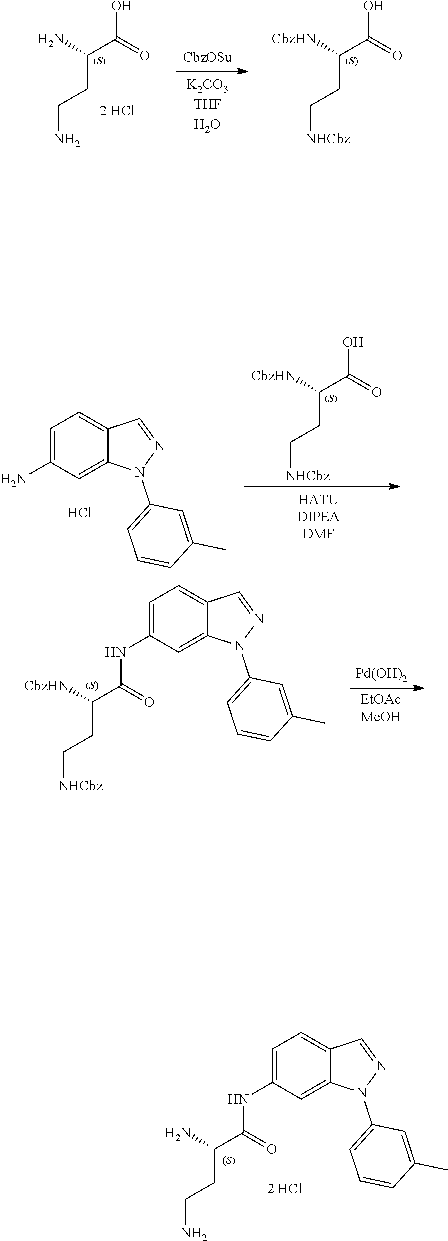

Step 1: To a mixture of (S)-2,4-diaminobutanoic acid dihydrochloride (500 mg, 2.62 mmol) and N-(benzyloxycarbonyloxy)succinimide (1.2 g, 4.82 mmol) in 4:1 THF:H₂O (25 mL) was added K₂CO₃ (1.3 g, 9.41 mmol). The resultant suspension was stirred for 15 h. The reaction mixture was concentrated to remove THF and then diluted with additional water (20 mL). The aqueous solution was washed with EtOAc (2×20 mL). The organic washes were discarded. The aqueous layer was acidified with 2 N HCl and then extracted into EtOAc (2×20 mL). This second organic layer was dried over Na₂SO₄, filtered, and concentrated to give (S)-2,4-bis(((benzyloxy)carbonyl)amino)butanoic acid as an off-white, gummy solid (650 mg, 64% yield). LC/MS and NMR are consistent.

Step 2: To a solution of 1-(m-tolyl)-1H-indazol-6-amine hydrochloride (75 mg, 0.29 mmol), (S)-2,4-bis(((benzyloxy)carbonyl)amino)butanoic acid (100 mg, 0.26 mmol), and HATU (130 mg, 0.34 mmol) in DMF (8 mL) at 0° C. was added DIPEA (113 μL, 0.65 mmol). The reaction mixture was stirred for 16 h, slowly coming to room temperature. LC/MS indicated the desired amide compound was formed. The reaction mixture was diluted with a saturated NaHCO₃ solution (40 mL) and extracted into EtOAc (100 mL). The organic layer was rinsed with a brine solution (2×30 mL). The organic layer was then dried over Na₂SO₄, filtered, and concentrated to give a crude oil. The crude was purified by CombiFlash chromatography (60% EtOAc:heptane) to give (S)-dibenzyl (4-oxo-4-((1-(m-tolyl)-1H-indazol-6-yl)amino)butane-1,3-diyl)dicarbamate as a gummy, off-white solid (120 mg, 78% yield). LC/MS and NMR are consistent.

Step 3: (S)-dibenzyl (4-oxo-4-((1-(m-tolyl)-1H-indazol-6-yl)amino)butane-1,3-diyl)dicarbamate (120 mg, 0.20 mmol) was dissolved in EtOAc in a hydrogenation flask. The flask was charged with Pd(OH)₂ (20 mg) and the flask was sealed and pressurized with H₂ to 45 psi. The mixture was stirred under pressure for 15 h at which point LC/MS indicated a complete deprotection. The reaction mixture was filtered through a bed of celite and the celite cake was rinsed with additional EtOAc (25 mL). The filtrate was concentrated to give a crude residue. TLC indicated 3 spots were present. The crude was purified by CombiFlash chromatography (5% MeOH:DCM, with 0.1% methanolic ammonia) to give the desired freebase. The freebase was dissolved in 1:1 MTBE:MeOH (2 mL) and treated with 2 N ethereal HCl (1 mL). The resultant suspension was concentrated to dryness and further dried under high vacuum to give (S)-2,4-diamino-N-(1-(m-tolyl)-1H-indazol-6-yl)butanamide dihydrochloride as a light brown solid (13 mg, 16% yield). LC/MS and NMR are consistent.

### Preparation of Compound 14

Compound 14 was prepared in a similar manner to compound 13.

### Preparation of Compound 15

Compound 15 was prepared in a similar manner to compound 13.

### Preparation of Compound 16

Compound 16 was prepared in a similar manner to compound 13.

### Preparation of Compound 17

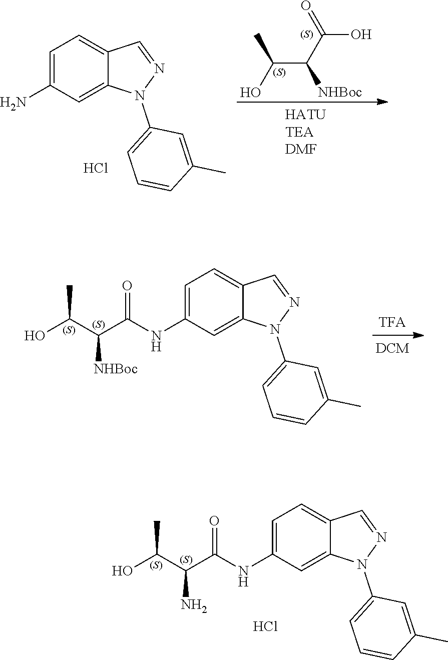

Step 1: To a solution of 1-(m-tolyl)-1H-indazolyl-6-amine hydrochloride (100 mg, 0.39 mmol) and HATU (190 mg, 0.50 mmol) in DMF (4 mL) was added dropwise a solution of (2S,3S)-2-((tert-butoxycarbonyl)amino)-3-hydroxybutanoic acid (110 mg, 0.50 mmol) and TEA (134 μL, 0.96 mmol) in DMF (4 mL) at 0° C. over 5 min. The reaction was stirred for 3.5 h at which point LC/MS indicated a clean and complete coupling. The reaction solution was diluted with DI H₂O (25 mL) and EtOAc (25 mL). The organic layer was then rinsed with a saturated NaHCO₃ solution (25 mL) and a brine solution (3×25 mL). The organic layer was dried over Na₂SO₄, filtered, and concentrated to give a crude oil. The crude was purified by CombiFlash chromatography (30% EtOAc:heptane) to give tert-butyl ((2S,3S)-3-hydroxy-1-oxo-1-((1-(m-tolyl)-1H-indazol-6-yl)amino)butan-2-yl)carbamate as a fluffy, pink solid (160 mg, 98% yield). NMR and LC/MS are consistent.

Step 2: To a solution of tert-butyl ((2S,3S)-3-hydroxy-1-oxo-1-((1-(m-tolyl)-1H-indazol-6-yl)amino)butan-2-yl)carbamate (160 mg, 0.38 mmol) in DCM (4 mL) at 0° C. was added TFA (1 mL). The mixture was stirred for 4 h, slowly coming to room temperature. LC/MS indicated a clean and complete deprotection. The reaction solution was concentrated to dryness and MTBE was added to the reaction vial. The mixture was treated with 2 N ethereal HCl and the resultant suspension was stirred for 30 min. The suspension was settled and the supernatant was decanted. The solid was resuspended in MTBE, let settle, and again decanted. The solid was then dried under high vacuum for 18 h. NMR indicated MTBE was present in the solid matrix of the salt. The solid was placed in a vacuum over for 3 days at 55° C. to give (2S,3S)-2-amino-3-hydroxy-N-(1-(m-tolyl)-1H-indazol-6-yl)butanamide hydrochloride as a tan solid (130 mg, 96% yield). NMR and LC/MS are consistent.

### Preparation of Compound 18

Compound 18 was prepared in a similar manner to compound 17.

### Preparation of Compound 19

Compound 19 was prepared in a similar manner to compound 17.

### Preparation of Compound 20

Compound 20 was prepared in a similar manner to compound 17.

### Preparation of Compound 21

Compound 21 was prepared in a similar manner to compound 17.

### Preparation of Compound 22

Compound 22 was prepared in a similar manner to compound 17.

### Preparation of Compound 23

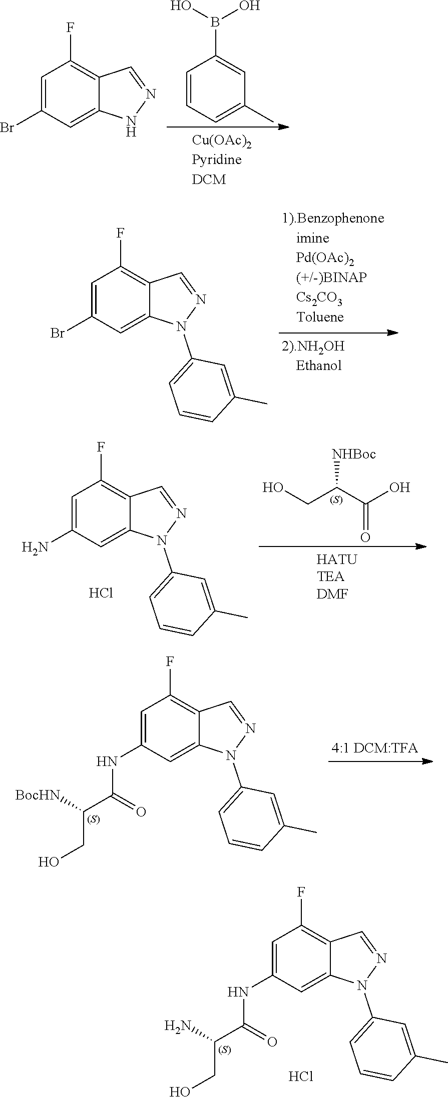

Step 1: 6-Bromo-4-fluoro-1H-indazole (5.0 g, 23.25 mmol), m-tolylboronic acid (3.8 g, 27.95 mmol), Cu(OAc)₂ (6.3 g, 34.69 mmol) and pyridine (3.9 mL,) were combined in DCM (250 mL). The resultant suspension was stirred for 16 h at which point LC/MS indicated a complete conversion to the desired compound. The reaction mixture was washed with a saturated NH₄Cl solution (3×150 mL). The organic layer was then dried over Na₂SO₄, filtered, and concentrated to give a red crude oil. The oil was purified by flash column (5% EtOAc:hexanes) to give 6-bromo-4-fluoro-1-(m-tolyl)-1H-indazole as a red oil (6.4 g, 90% yield). LC/MS and NMR are consistent. NMR shows less than 10% of the N2 isomer is present. No further purification attempted.

Step 2: 6-Bromo-4-fluoro-1-(m-tolyl)-1H-indazole (2.8 g, 9.18 mmol), benzophenone imine (2.1 g, 11.59 mmol), (+/−)-BINAP (280 mg, 0.45 mmol), Cs₂CO₃ (6.0 g, 18.42 mmol), and toluene (50 mL) were combined in a glass bomb. The mixture was purged with N₂ for 15 min and then Pd(OAc)₂ (210 mg, 0.94 mmol) was added. The mixture was purged with N₂ for an additional 3 min. The vessel was then sealed and the reaction mixture was heated to 100° C. for 18 h. LC/MS indicated a complete conversion to the imine. The reaction mixture was diluted with EtOAc (50 mL) and filtered through a thin bed of celite. The celite was rinsed with an additional 50 mL of EtOAc. The filtrate was concentrated to dryness. This crude was taken on to the next step without further purification.

Step 3: The crude material from the previous step was dissolved in EtOH (10 mL) and an aqueous hydroxylamine solution (338 μL, 46.7 mmol). The reaction mixture was stirred at room temperature for 5 h at which point LC/MS indicated a complete hydrolysis to the desired amino compound, but showed several side products by TLC (total 4 spots). The reaction mixture was concentrated in vacuo. The resultant syrup was dissolved in DCM (30 mL) and washed with water (30 mL). The organic layer was dried over Na₂SO₄, filtered, and concentrated to give a crude red oil. The crude was purified by column chromatography (10% EtOAc:heptane) to give the pure free base as a red oil. The free base was dissolved in MTBE (8 mL) and 2 N ethereal HCl (2 mL) was added forming an HCl salt. The suspension was filtered, rinsed with excess MTBE (5 mL) and air dried for 10 min to give 4-fluoro-1-(m-tolyl)-1H-indazol-6-amine hydrochloride as a pale pink solid (810 mg, 32% yield over two steps). NMR and LC/MS are consistent.

Step 4: To a solution of 4-fluoro-1-(m-tolyl)-1H-indazol-6-amine hydrochloride (150 mg, 0.54 mmol) and HATU (270 mg, 0.71 mmol) in DMF (5 mL) at 0° C. was added a solution of L-N-Cbz serine (150 mg, 0.63 mmol) and TEA (188 μL, 1.35 mmol) in DMF (3 mL) dropwise over 3 min. The solution was stirred for 18 h, slowly coming to room temperature. LC/MS indicated a clean conversion. The reaction mixture was diluted with water (20 mL) and extracted into EtOAc (25 mL). The organic layer was rinsed with 1 N HCl (20 mL), a saturated NaHCO₃ solution (20 mL), and a brine solution (2×10 mL). The organic layer was then dried over Na₂SO₄, filtered, and concentrated to give a crude oil. The crude material was purified by CombiFlash chromatography (50% EtOAc:heptane) to give (S)-tert-butyl (1-((4-fluoro-1-(m-tolyl)-1H-indazol-6-yl)amino)-3-hydroxy-1-oxopropan-2-yl)carbamate as a pale pink solid (220 mg, 95% yield). LC/MS is clean and consistent.

Step 5: To a solution of (S)-tert-butyl (1-((4-fluoro-1-(m-tolyl)-1H-indazol-6-yl)amino)-3-hydroxy-1-oxopropan-2-yl)carbamate (220 mg, 0.51 mmol) in DCM (4 mL) at 0° C. was added TFA (1 mL). The mixture was stirred for 4.5 h, slowly coming to room temperature. LC/MS indicated a clean and complete deprotection. The reaction solution was concentrated to dryness and MTBE was added to the reaction vial. The mixture was treated with 2 N ethereal HCl and the resultant suspension was stirred for 30 min and filtered to give (S)-2-amino-N-(4-fluoro-1-(m-tolyl)-1H-indazol-6-yl)-3-hydroxypropanamide hydrochloride as a brown solid (130 mg, 70%). LC/MS and NMR are consistent.

### Preparation of Compound 24

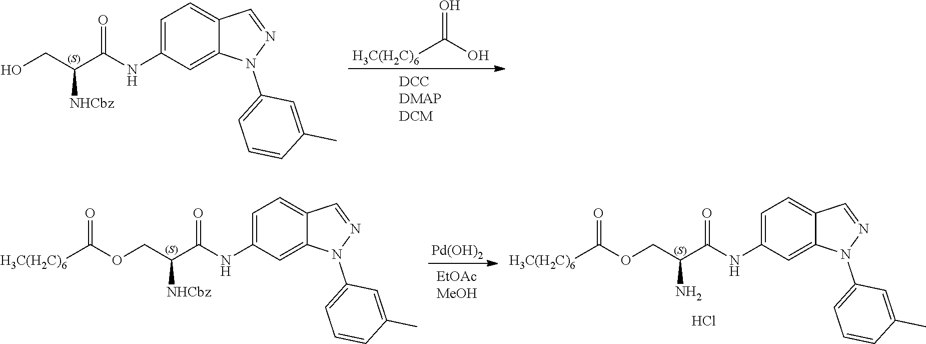

Step 1: (S)-benzyl (3-hydroxy-1-oxo-1-((1-(m-tolyl)-1H-indazol-6-yl)amino)propan-2-yl)carbamate (380 mg, 0.85 mmol), octanoic acid (240 mg, 1.66 mmol), DCC (230 mg, 1.11 mmol), and DMAP (90 mg, 0.74 mmol) were combined in DCM and stirred at room temperature for 20 h. LC/MS showed a clean and complete coupling to the desired ester. The reaction mixture was filtered to removed insoluble DCC byproducts and then concentrated to give a crude oil. The crude material was purified by CombiFlash chromatography (15% EtOAc:heptane) to give (S)-2-(((benzyloxy)carbonyl)amino)-3-oxo-3-((1-(m-tolyl)-1H-indazol-6-yl)amino)propyl octanoate as a white solid (330 mg, 68% yield). LC/MS and NMR are consistent.

Step 2: To a solution of (S)-2-(((benzyloxy)carbonyl)amino)-3-oxo-3-((1-(m-tolyl)-1H-indazol-6-yl)amino)propyl octanoate (330 mg, 0.58 mmol) in EtOH (10 mL) was added Pd(OH)₂ (50 mg). The reaction vessel was pressurized to 60 psi with H₂. The reaction mixture was stirred under pressure for 20 h. At which point LC/MS indicated a complete deprotection. The mixture was filtered through a bed of celite, the celite cake was rinsed with additional EtOH (10 mL), and the filtrate was concentrated. Minor side products formed during the deprotection so the desired freebase was isolated by CombiFlash chromatography (15% EtOAc:heptane). The freebase was dissolved in EtOAc (with a drop of MeOH for solubility). The solution was treated with 2 N ethereal HCl, the mixture was concentrated under rotary evaporation, and the resulting solid was further dried under high vacuum for 18 h to give (S)-2-amino-3-oxo-3-((1-(m-tolyl)-1H-indazol-6-yl)amino)propyl octanoate hydrochloride as a tan solid (185 mg, 68% yield). NMR and LC/MS are consistent.

### Preparation of Compound 25

Compound 25 was prepared in a similar manner to compound 24.

### Preparation of Compound 26

Compound 26 was prepared in a similar manner to compound 24.

### Preparation of Compound 27

Compound 27 was prepared in a similar manner to compound 24.

### Preparation of Compound 28

Compound 28 was prepared in a similar manner to compound 24.

### Preparation of Compound 29

Compound 29 was prepared in a similar manner to compound 24.

### Preparation of Compound 30

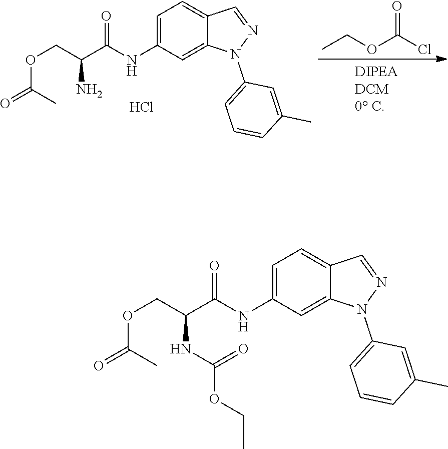

Step 1: To a solution of ABCL/CRP-099 (80 mg, 0.21 mmol) and DIPEA (90 μL, 0.52 mmol) in DCM (3 mL) at 0° C. was added ethyl chloroformate (22 μL, 0.23 mmol). The mixture was stirred for 4 h at which point LC/MS showed that the desired carbamate was formed. The mixture was diluted with additional DCM (10 mL) and rinsed with a saturated NaHCO₃ solution (10 mL). The organic layer was dried over Na₂SO₄, filtered, and concentrated. The resultant residue was purified by CombiFlash chromatography (30% EtOAc:heptane) to give (S)-2-((ethoxycarbonyl)amino)-3-oxo-3-((1-(m-tolyl)-1H-indazol-6-yl)amino)propyl acetate as an off-white solid (50 mg, 57% yield). LC/MS and NMR are consistent.

### Preparation of Compound 31

Compound 31 was prepared in a similar manner to compound 30.

### Preparation of Compound 32

Compound 32 was prepared in a similar manner to compound 30.

### Preparation of Compound 33

Compound 33 was prepared in a similar manner to compound 30.

### Preparation of Compound 34

Compound 34 was prepared in a similar manner to compound 30.

### Preparation of Compound 35

Compound 35 was prepared in a similar manner to compound 30.

### Preparation of Compound 36

Compound 36 was prepared in a similar manner to compound 30.

### Preparation of Compound 37

Compound 37 was prepared in a similar manner to compound 30.

### Preparation of Compound 38

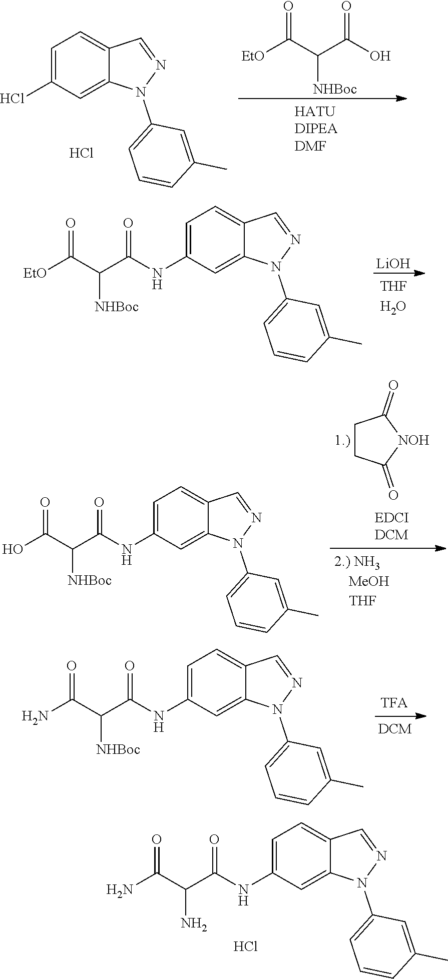

Step 1: To a solution of 1-(m-tolyl)-1H-indazol-6-amine hydrochloride (300 mg, 1.15 mmol), 2-((tert-butoxycarbonyl)amino)-3-ethoxy-3-oxopropanoic acid (344 mg, 1.39 mmol), and HATU (662 mg, 1.74 mmol) in DMF (5 mL) at 0° C. was added DIPEA (624 μL, 3.58 mmol). The reaction mixture was stirred for 16 h, slowly coming to room temperature. The reaction mixture was diluted with H₂O (20 mL) and extracted into EtOAc (25 mL). The organic layer was rinsed with a saturated NaHCO₃ solution (20 mL) and then a brine solution (2×20 mL). The organic layer was then dried over Na₂SO₄, filtered, and concentrated to give a crude oil. The crude was purified by CombiFlash chromatography (50% EtOAc:heptane) to give ethyl 2-((tert-butoxycarbonyl)amino)-3-oxo-3-((1-(m-tolyl)-1H-indazol-6-yl)amino)propanoate as a white solid (300 mg, 57% yield). LC/MS is consistent.

Step 2: A solution of ethyl 2-((tert-butoxycarbonyl)amino)-3-oxo-3-((1-(m-tolyl)-1H-indazol-6-yl)amino)propanoate (300 mg, 0.66 mmol) in 4:1 THF:H₂O (25 mL) was treated with LiOH.H₂O (48 mg, 1.14 mmol). The resultant suspension was stirred for 18 h and then treated with 2 N HCl (10 mL). The THF was removed by rotary evaporation and the mixture was extracted into EtOAc (20 mL). The organic layer was dried over Na₂SO₄, filtered, and concentrated to get a crude solid. The solid was recrystallized using 2:1 DCM:hexanes to give 2-((tert-butoxycarbonyl)amino)-3-oxo-3-((1-(m-tolyl)-1H-indazol-6-yl)amino)propanoic acid (230 mg, 82% yield). LC/MS and NMR are consistent.

Step 3: To a solution of 2-((tert-butoxycarbonyl)amino)-3-oxo-3-((1-(m-tolyl)-1H-indazol-6-yl)amino)propanoic acid (230 mg, 0.54 mmol) in DCM (5 mL) was added N-hydroxysuccinimide (75 mg, 0.65 mmol) and EDCl (126 mg, 0.81 mmol). The reaction mixture was stirred for 2 h. The mixture was diluted with additional DCM (10 mL) and H₂O (10 mL). The organic layer was dried over Na₂SO₄, filtered, and concentrated to give a crude oil. The crude material was purified by CombiFlash chromatography (50% EtOAc:heptane) to give the desired succinimide ester. The succinimide ester was dissolved in THF (5 mL) and was treated with excess of methanolic ammonia. The reaction solution was stirred for 1 h and then concentrated to give a crude oil. The crude was purified by CombiFlash chromatography (75% EtOAc:heptane) to give tert-butyl (1-amino-1,3-dioxo-3-((1-(m-tolyl)-1H-indazol-6-yl)amino)propan-2-yl)carbamate as an off-white solid (125 mg, 55% yield over two steps). LC/MS and NMR are consistent.

Step 4: A solution of tert-butyl (3-hydroxy-2-(1-(m-tolyl)-1H-indazole-6-carboxamido)propyl)carbamate (120 mg, 0.28 mmol) in DCM (4 mL) was treated with TFA (2 mL). The solution was stirred for 2 h at which point TLC indicated the starting Boc compound was consumed. The reaction solution was concentrated to dryness to give the desired freebase. The freebase was dissolved in MTBE (3 mL with a drop of MeOH for solubility) and treated with 2 N ethereal HCl (1 mL). The resultant suspension was filtered to give 2-amino-N¹-(1-(m-tolyl)-1H-indazol-6-yl)malonamide hydrochloride as a white solid (51 mg, 50% yield). LC/MS and NMR are consistent.

### Preparation of Compound 39

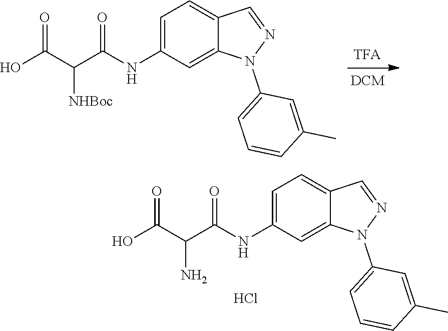

A solution of 2-((tert-butoxycarbonyl)amino)-3-oxo-3-((1-(m-tolyl)-1H-indazol-6-yl)amino)propanoic acid (100 mg, 0.24 mmol) in DCM (8 mL) was treated with TFA (2 mL). The solution was stirred for 2 h at which point TLC indicated the starting Boc compound was consumed. The reaction solution was concentrated to dryness to give the desired freebase. The freebase was dissolved in MTBE (3 mL) treated with 2 N ethereal HCl (1 mL) and the resultant salt filtered and further dried under high vacuum to 2-amino-3-oxo-3-((1-(m-tolyl)-1H-indazol-6-yl)amino)propanoic acid hydrochloride as a white solid (55 mg, 72% yield). LC/MS and NMR are consistent.

### Preparation of Compound 40

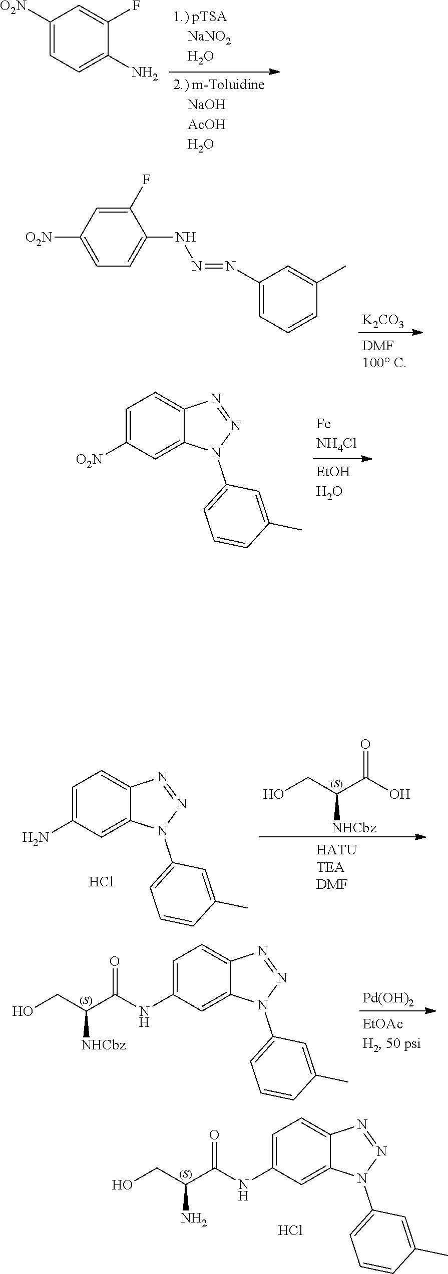

Step 1: In a mortar and pestle 2-Fluoro-4-nitroaniline (0.47 g, 3.0 mmol) and p-toluene sulfonic acid (1.71 g, 9.0 mmol) were ground together with 1 mL of water for 3 min forming a bright yellow paste. Then NaNO₂ (0.41 g, 5.9 mmol) was added and the mixture was ground together for a further 10 min. The resultant paste was diluted with 3 mL of water. To that paste was added a chilled slurry of m-toluidine (0.43 g, 4.0 mmol), NaOH (8 g, 200 mmol), AcOH (12 mL, 200 mmol) in 2 mL water. The resultant mixture was a deep purple slurry. The mixture was ground continuously for 1 h and then diluted with an additional 5 mL of chilled water and filtered. LC/MS showed a mixture of compounds but the desired triazene was the major component of the mixture. The filtrand was dried for 2 h under vacuum suction to give (3-(2-fluoro-4-nitrophenyl)-1-(m-tolyl)triaz-1-ene as a crude, black solid (8 g). Yield not calculated. No NMR data was taken. Taken on to cyclization step without further purification.

Step 2: To a solution of crude 3-(2-fluoro-4-nitrophenyl)-1-(m-tolyl)triaz-1-ene (8 g, 29.2 mmol) in DMSO (300 mL) was added K₂CO₃ (12.1 g, 87.5 mmol). The reaction mixture was heated to 100° C. for 16 h at which point LC/MS indicated the cyclization was complete. The reaction mixture was diluted with chilled water (30 mL), stirred for 10 min, and then filtered. The crude filtrand was air dried for 30 min then dissolved in EtOAc, dried over Na₂SO₄, filtered, and concentrated. The crude was then purified by column chromatography (10% EtOAc:heptane) to give 6-nitro-1-(m-tolyl)-1H-benzo[d][1,2,3]triazole as a tan solid (0.64 g, 8% yield over two steps). LC/MS and NMR are consistent.

Step 3: To a suspension of 6-nitro-1-(m-tolyl)-1H-benzo[d][1,2,3]triazole (750 mg, 2.95 mmol) in 5:1 EtOH:H₂O (25 mL) was added Fe powder (500 mg, 8.95 mmol), and NH₄Cl (790 mg, 14.77 mmol). The mixture was heated to 85° C. for 1.5 h at which point LC/MS indicated a complete conversion to the amino compound. The mixture was filtered through a celite bed and the celite was rinsed with additional 25 mL of EtOH. The filtrate was concentrated, diluted with an additional 10 mL of H₂O, and extracted into EtOAc (25 mL). The organic layer was dried over Na₂SO₄, filtered, and concentrated to give a dark, crude oil. The crude was purified by CombiFlash chromatography (30% EtOAc:heptane) to give a dark oil. NMR showed a clean freebase. The oil was dissolved in MTBE (3 mL), treated with 2 N ethereal HCl (2 mL) then concentrated to dryness, and further dried under high vacuum to give 6-amino-1-(m-tolyl)-1H-benzo[d][1,2,3]triazole hydrochloride as a beige solid (530 mg, 68.9% yield). LC/MS and NMR are consistent.

Step 4: To a solution of 1-(m-tolyl)-1H-benzo[d][1,2,3]triazol-6-amine hydrochloride (100 mg, 0.38 mmol) and HATU (190 mg, 0.50 mmol) in DMF (4 mL) at 0° C. was added a solution of L-Cbz serine (120 mg, 0.50 mmol) and TEA (134 μL, 0.96 mmol) in DMF (4 mL) dropwise over 5 min. The solution was stirred for 15 h at which point LC/MS indicated a clean coupling. The mixture was diluted with a saturated NaHCO₃ solution (40 mL) and extracted into EtOAc (100 mL). The organic layer was rinsed with a brine solution (2×30 mL). The organic layer was then dried over Na₂SO₄, filtered, and concentrated. The oil was purified by CombiFlash chromatography (50% EtOAc:heptane) to give (S)-benzyl (3-hydroxy-1-oxo-1-((1-(m-tolyl)-1H-benzo[d][1,2,3]triazol-6-yl)amino)propan-2-yl)carbamate as a pale yellow residue (150 mg, 88% yield). LC/MS and NMR are consistent.

Step 5: To a solution of (S)-benzyl (3-hydroxy-1-oxo-1-((1-(m-tolyl)-1H-benzo[d][1,2,3]triazol-6-yl)amino)propan-2-yl)carbamate (150 mg, 0.34 mmol) in EtOAc (10 mL) was added Pd(OH)₂ (30 mg). The reaction vessel was pressurized with H₂ to 50 psi and the mixture was stirred for 16 h. LC/MS indicated a complete deprotection. The reaction mixture was filtered through a bed of celite and the celite cake was rinsed with an additional 30 mL of EtOAc. The filtrate was concentrated to give clean freebase. The freebase was dissolved in MTBE with a few drops of MeOH and treated with 2 N ethereal HCl. The resultant suspension was concentrated to dryness and the solid was further dried under high vacuum for 6 h to give (S)-2-amino-3-hydroxy-N-(1-(m-tolyl)-1H-benzo[d][1,2,3]triazol-6-yl)propanamide hydrochloride as a tan solid (60 mg, 51% yield). The NMR and LC/MS are consistent.

### Preparation of Compound 41

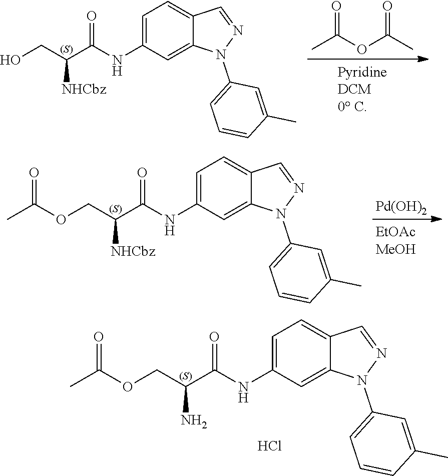

Step 1: To a suspension of (S)-benzyl (3-hydroxy-1-oxo-1-((1-(m-tolyl)-1H-indazol-6-yl)amino)propan-2-yl)carbamate (75 mg, 0.17 mmol) and pyridine (21 μL, 0.26 mmol) in DCM (2 mL) at 0° C. was added Ac₂O (63 μL, 0.67 mmol). The mixture was stirred for 15 h, slowly coming to room temperature. LC/MS indicated that the starting alcohol was completely consumed and the desired product was formed. The mixture was diluted with additional DCM (20 mL) and rinsed with a 2 N HCl solution (20 mL), a saturated NaHCO₃ solution (20 mL), and a brine solution (20 mL). The organic layer was dried over Na₂SO₄ and filtered. The filtrate was directly absorbed to silica gel and the product was purified by CombiFlash chromatography (30% EtOAc:heptane) to give (S)-2-(((benzyloxy)carbonyl)amino)-3-oxo-3-((1-(m-tolyl)-1H-indazol-6-yl)amino)propyl acetate as a white solid (55 mg, 67% yield). LC/MS is consistent and very clean. NMR not taken.

Step 2: To a solution of (S)-2-(((benzyloxy)carbonyl)amino)-3-oxo-3-((1-(m-tolyl)-1H-indazol-6-yl)amino)propyl acetate (50 mg, 0.11 mmol) was added Pd(OH)₂ (20 mg). The reaction vessel was pressurized to 50 psi with H₂ and stirred for 18 h. LC/MS showed a clean and complete deprotection. The reaction mixture was filtered through a small bed of celite and the celite cake was rinsed with additional EtOAc (50 mL). The filtrate was concentrated to dryness, redissolved in 3 mL of EtOAc, and treated with 2 N ethereal HCl. The resultant suspension was concentrated to dryness and further dried under high vacuum for 12 h to give (S)-2-amino-3-oxo-3-((1-(m-tolyl)-1H-indazol-6-yl)amino)propyl acetate hydrochloride as an off-white solid (44 mg, 100% yield). NMR and LC/MS are consistent.

### Preparation of Compound 42

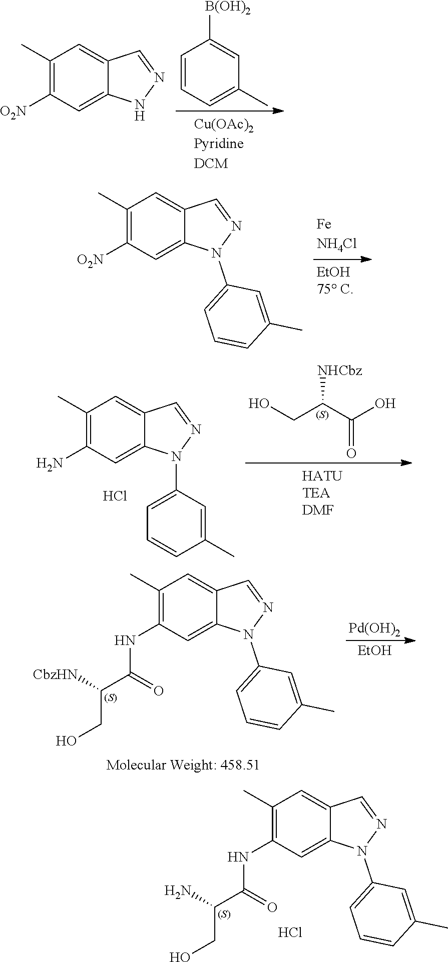

Step 1: A suspension of 5-methyl-6-nitro-1H-indazole (2.0 g, 11.30 mmol), m-tolylboronic acid (1.8 g, 13.23 mmol), Cu(OAc)₂ (3.0 g, 16.52 mmol), and pyridine (2.7 mL, 33.52 mmol) in DCM (20 mL) was stirred for 16 h. LC/MS indicated a clean conversion. The reaction mixture was directly absorbed to silica gel and purified by column chromatography (10% EtOAc:heptane) to give methyl-6-nitro-1-(m-tolyl)-1H-indazole as a 1:1 mixture of N1 and N2 isomers (1.8 g, 60% yield). Isomeric ratio determined by NMR. Compound is a white solid. LC/MS is consistent.

Step 2: A suspension of 5-methyl-6-nitro-1-(m-tolyl)-1H-indazole (1.5 g, 5.61 mmol), Fe powder (0.9 g, 16.11 mmol), and NH₄Cl (1.6 g, 28.83 mmol) in 5:1 EtOH:H₂O (60 mL) was heated to 75° C. for 2 h. TLC indicated a complete conversion. The reaction mixture was cooled to room temperature, filtered through a bed of celite, and the celite cake was rinse with additional EtOH (20 mL). The filtrate was concentrated to remove EtOH, diluted with additional H₂O (20 mL), and extracted into EtOAc (3×30 mL). The organic layer was dried over Na₂SO₄, filtered, and concentrated to give a crude oil. The crude was purified by CombiFlash chromatography (30% EtOAc:heptane) to give the desired freebase. The freebase was dissolved in EtOAc (10 mL) and treated with 2 N ethereal HCl. The resultant suspension was stirred for 30 min and then filtered to give 5-methyl-1-(m-tolyl)-1H-indazole-6-amine hydrochloride as an off-white solid (600 mg, 39% yield). LC/MS and NMR are consistent.

Step 3: To a solution of 1-(m-tolyl)-1H-indazol-6-amine hydrochloride (150 mg, 0.55 mmol) and HATU (270 g, 0.71 mmol) in DMF (4 mL) at 0° C. was added a solution of L-N-Cbz serine (170 mg, 0.71 mmol) and TEA (191 μL, 1.37 mmol) in DMF (2 mL) dropwise over 3 min. The solution was stirred for 18 h, slowly coming to room temperature. LC/MS indicated a clean conversion. The reaction mixture was diluted with a saturated NaHCO₃ solution (20 mL) and extracted into EtOAc (20 mL). The organic layer was rinsed with a brine solution (3×10 mL). The organic layer was then dried over Na₂SO₄, filtered, and concentrated to give a crude amber oil. The crude was purified by CombiFlash chromatography (50% EtOAc:heptane) to give (S)-benzyl (3-hydroxy-1-((5-methyl-1-(m-tolyl)-1H-indazol-6-yl)amino)-1-oxopropan-2-yl)carbamate as an off-white solid (180 mg, 71% yield). LC/MS and NMR of product are consistent.

Step 4: To a solution (S)-benzyl (3-hydroxy-1-((5-methyl-1-(m-tolyl)-1H-indazol-6-yl)amino)-1-oxopropan-2-yl)carbamate (180 mg, 0.39 mmol) in EtOH (10 mL) was added Pd(OH)₂ (50 mg). The reaction vessel was pressurized to 60 psi with H₂. The reaction mixture was stirred under pressure for 18 h. At which point LC/MS indicated a complete deprotection. The freebase was dissolved in EtOAc (with a drop of MeOH for solubility). The solution was treated with 2 N ethereal HCl and the resultant suspension was stirred for 20 min. The suspension was filtered and the solid was dried under high vacuum for 24 h to give (S)-2-amino-3-hydroxy-N-(5-methyl-1-(m-tolyl)-1H-indazol-6-yl)propanamide hydrochloride as an off-white solid (65 mg, 55% yield). NMR and LC/MS are consistent.

### Preparation of Compound 43

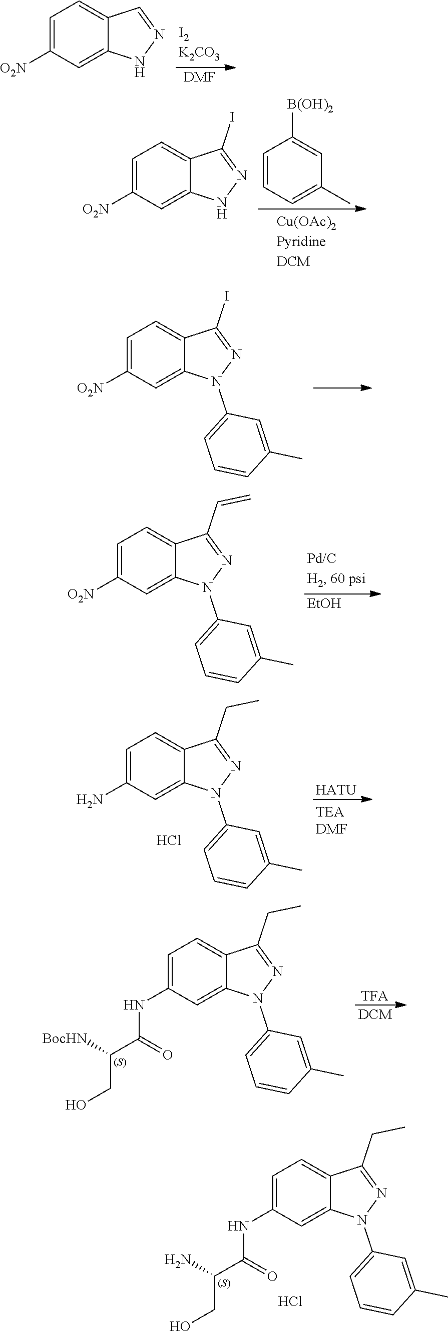

Step 1: To a solution of 6-nitroindazole (2.00 g, 12.26 mmol) in DMF (10 mL) was added K₂CO₃ (3.38 g, 2.0 equiv) while maintaining a temperature below 30° C. A solution of iodine (5.28 g, 1.7 equiv) was added over 1 h while maintaining a temperature below 30° C. After 3 h, LC/MS indicated a clean conversion. To the reaction mixture was then added a solution of sodium thiosulfate (3.0 g, 12.09 mmol) and potassium carbonate (0.23 g, 1.7 mmol) while maintaining a solution temperature below 30° C. The mixture was stirred for 20 min. H₂O (30 mL) was then added forming a thick suspension. The suspension was stirred an additional 20 min and then filtered. The filtrand was dried in a vacuum oven at 50° C. over 36 h to give 3-iodo-6-nitro-1H-indazole as an off-white solid (3.4 g, 97% yield). LC/MS and NMR are consistent.

Step 2: A suspension of 3-iodo-6-nitro-1H-indazole (4.1 g, 14.19 mmol), m-tolylboronic acid (2.5 g, 18.39 mmol), Cu(OAc)₂ (3.9 g, 21.47 mmol), and pyridine (2.3 mL, 28.55 mmol) in DCM (100 mL) was stirred for 16 h, at which point LC/MS indicated a clean conversion to the desired compound. The mixture was filtered and the filtrate was concentrated. The resultant crude was run through a silica plug (10% EtOAc:heptane) to give 3-iodo-6-nitro-1-(m-tolyl)-1H-indazole as a yellow solid as a 10:1 mixture of N1 and N2 isomers (4.2 g, 78% yield). NMR and LC/MS are consistent.

Step 3: A suspension of 3-iodo-6-nitro-1-(m-tolyl)-1H-indazole (600 mg, 1.58 mmol) and potassium trifluoro(vinyl)borate (670 mg, 5.00 mmol) was bubbled with N₂ for 20 min. TEA (662 μL, 4.99 mmol) and Pd(dppf)Cl₂ DCM (130 mg) were added. The reaction vessel was sealed and heated to 100° C. for 3.5 h. LC/MS showed the complete consumption of the lodo compound. The reaction mixture was diluted with EtOAc and filtered through celite. The filtrate was concentrated to give a crude reddish brown solid. The crude was purified by CombiFlash chromatography (5% EtOAc:heptane) to give 6-nitro-1-(m-tolyl)-3-vinyl-1H-indazole as a yellow solid (260 mg, 59% yield). LC/MS and NMR are consistent.

Step 4: A suspension of 3-iodo-6-nitro-1-(m-tolyl)-1H-indazole (1.80 g, 4.74 mmol) and potassium trifluoro(vinyl)borate (1.94 g, 14.48 mmol) in 4:1 IPA:THF (15 mL) was bubbled with N₂ for 20 min. TEA (1.98 mL, 14.21 mmol) and Pd(dppf)Cl₂ DCM (390 mg) were added. The reaction vessel was sealed and heated to 100° C. for 3.5 h. LC/MS showed the complete consumption of the lodo compound. The reaction mixture was diluted with EtOAc and filtered through celite. The filtrate was concentrated to give a crude reddish brown solid. The crude was purified by CombiFlash chromatography (5% EtOAc:heptane) to give 6-nitro-1-(m-tolyl)-3-vinyl-1H-indazole as a yellow solid (670 mg, 50% yield). LC/MS and NMR are consistent.

Step 5: A solution of 6-nitro-1-(m-tolyl)-3-vinyl-1H-indazole (670 mg, 2.40 mmol) in EtOH (20 mL) in a hydrogenation flask was charged with 10% Pd/C, wet (80 mg). The flask was sealed and pressurized to 60 psi with H₂. The reaction mixture was stirred under pressure for 18 h. At which point LC/MS indicated a ˜90% conversion to the desired product. The mixture was filtered through a bed of celite and the celite cake was rinsed with additional EtOH (20 mL). The filtrate was concentrated to dryness to give a crude oil. The crude was purified by CombiFlash chromatography (30% EtOAc:heptane) to give the desired freebase. The freebase was dissolved in EtOAc and the solution was treated with 2 N ethereal HCl. The resultant suspension was stirred for 20 min and then it was filtered to give 3-ethyl-1-(m-tolyl)-1H-indazol-6-amine hydrochloride as an off-white solid (580 mg, 84% yield). LC/MS and NMR are consistent.

Step 6: To a solution of 3-ethyl-1-(m-tolyl)-1H-indazol-6-amine hydrochloride (130 mg, 0.42 mmol) and HATU (220 mg, 0.58 mmol) in DMF (4 mL) at 0° C. was added a solution of L-N-Boc-serine (120 mg, 0.58 mmol) and TEA (145 μL, 1.04 mmol) in DMF (2 mL) dropwise over 3 min. The solution was stirred for 18 h, slowly coming to room temperature. LC/MS indicated a clean conversion. The reaction mixture was diluted with a saturated NaHCO₃ solution (20 mL) and extracted into EtOAc (20 mL). The organic layer was rinsed with a brine solution (3×20 mL). The organic layer was then dried over Na₂SO₄, filtered, and concentrated to give a crude solid. The crude solid was purified by CombiFlash chromatography (50% EtOAc:heptane) to give (S)-tert-butyl (1-((3-ethyl-1-(m-tolyl)-1H-indazol-6-yl)amino)-3-hydroxy-1-oxopropan-2-yl)carbamate as an off-white, gummy solid (160 mg, 81%). LC/MS and NMR are consistent.

Step 7: To a solution of (S)-tert-butyl (1-((3-ethyl-1-(m-tolyl)-1H-indazol-6-yl)amino)-3-hydroxy-1-oxopropan-2-yl)carbamate (160 mg, 0.36 mmol) in DCM (4 mL) was added TFA (1 mL). The mixture was stirred for 4 h at which point TLC indicated a complete deprotection. The reaction solution was concentrated to give an amber oil. The oil was redissolved in MTBE and treated with 2 N ethereal HCl. The resultant suspension was filtered and the solid dried under high vacuum to give (S)-2-amino-N-(3-ethyl-1-(m-tolyl)-1H-indazol-6-yl)-3-hydroxypropanamide hydrochloride as a pale pink solid (65 mg, 47% yield). LC/MS and NMR are consistent.

### Preparation of Compound 44

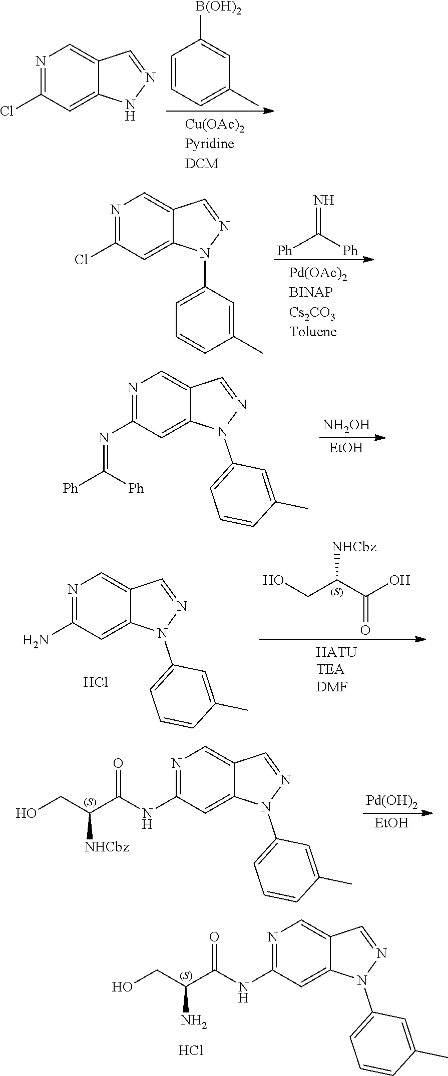

Step 1: 6-chloro-1H-pyrazolo[4,3-c]pyridine (500 mg, 3.26 mmol), m-tolylboronic acid (580 mg, 4.27 mmol), Cu(OAc)₂ (890 mg, 4.90 mmol), pyridine (525 μL, 6.52 mmol), and DCM (20 mL) were combined in a round bottom flask. The flask was fitted with a drying tube and stirred at room temperature for 16 h. TLC and LC/MS indicated a complete conversion. The reaction mixture was directly absorbed onto silica and purified by column chromatography (10% EtOAc:heptane) to give 6-chloro-1-(m-tolyl)-1H-pyrazolo[4,3-c]pyridine as a fluffy, white solid (750 mg, 95% yield). NMR shows a small amount of a regioisomer (˜10%). LC/MS is consistent. Taken on to next step without further purification.

Step 2: 6-chloro-1-(m-tolyl)-1H-pyrazolo[4,3-c]pyridine (570 mg, 2.34 mmol), benzophenone imine (530 mg, 2.92 mmol), Pd(OAc)₂ (50 mg, 0.22 mmol), (+/−)-BINAP (70 mg, 0.11 mmol), Cs₂CO₃ (1.52 g, 4.67 mmol) and toluene (10 mL) were combined in a glass bomb. The reaction mixture was bubbled with N₂ for 15 min and then the vessel sealed. The reaction mixture was heated to 100° C. for 15 h. LC/MS indicated a clean conversion to the imine. The reaction mixture was diluted with EtOAc (50 mL) and filtered through a thin bed of celite. The celite was rinsed with an additional 50 mL of EtOAc. The filtrate was concentrated to dryness to give crude N-(diphenylmethylene)-1-(m-tolyl)-1H-pyrazolo[4,3-c]pyridin-6-amine as an amber oil. The crude was dissolved in EtOH (15 mL) and hydroxylamine (1 mL) was added. The reaction mixture was stirred at room temperature for 3 h and then heated to 50° C. for 3 h. LC/MS indicated complete hydrolysis of the imine. The ethanol was concentrated to dryness giving a crude amber oil. The crude oil was dissolved in MTBE (10 mL) and then treated with 2 M ethereal hydrochloric acid (2 mL). The mixture was stirred overnight until a free flowing solid was formed. The suspension was filtered to give 1-(m-tolyl)-1H-pyrazolo[4,3-c]pyridin-6-amine hydrochloride as an off-white solid (330 mg, 54% over two steps). LC/MS and NMR were consistent.

Step 3: To a solution of 1-(m-tolyl)-1H-pyrazolo[4,3-c]pyridin-6-amine hydrochloride (150 mg, 0.58 mmol) and HATU (280 mg, 0.74 mmol) in DMF (4 mL) at 0° C. was added dropwise a solution of L-N-Cbz Serine (180 mg, 0.75 mmol) and TEA (200 μL, 1.43 mmol) in DMF (2 mL). The mixture was stirred for 72 h, slowly coming to room temperature. LC/MS indicated that the reaction only went to ˜20% completion. The reaction mixture was diluted with a saturated NaHCO₃ solution (20 mL) and extracted into EtOAc (20 mL). The organic layer was rinsed with a brine solution (3×10 mL). The organic layer was then dried over Na₂SO₄, filtered, and concentrated to give a crude amber oil. The crude was purified by CombiFlash chromatography (50% EtOAc:heptane) to give (S)-benzyl (3-hydroxy-1-oxo-1-((1-(m-tolyl)-1H-pyrazolo[4,3-c]pyridin-6-yl)amino)propan-2-yl)carbamate as a pale yellow residue (46 mg, 18% yield). NMR and LC/MS are consistent.

Step 4: To a solution (S)-benzyl (3-hydroxy-1-oxo-1-((1-(m-tolyl)-1H-pyrazolo[4,3-c]pyridin-6-yl)amino)propan-2-yl)carbamate (45 mg, 0.10 mmol) in EtOH (5 mL) was added Pd(OH)₂ (10 mg). The reaction vessel was sealed and pressurized to 60 psi with H₂. The reaction mixture was stirred under pressure for 18 h. At which point LC/MS indicated a complete deprotection. The freebase was dissolved in EtOAc (1 mL with a drop of MeOH for solubility). The solution was treated with 2 N ethereal HCl and the resultant suspension was stirred for 20 min. The suspension was filtered and dried under vacuum to give (S)-2-amino-3-hydroxy-N-(1-(m-tolyl)-1H-pyrazolo[4,3-c]pyridin-6-yl)propanamide hydrochloride as an yellow solid (8 mg, 23% yield). NMR and LC/MS are consistent.

### Preparation of Compound 45

Compound 45 was prepared in a similar manner to compound 44.

### Preparation of Compound 46

Compound 46 was prepared in a similar manner to compound 44.

### Preparation of Compound 47

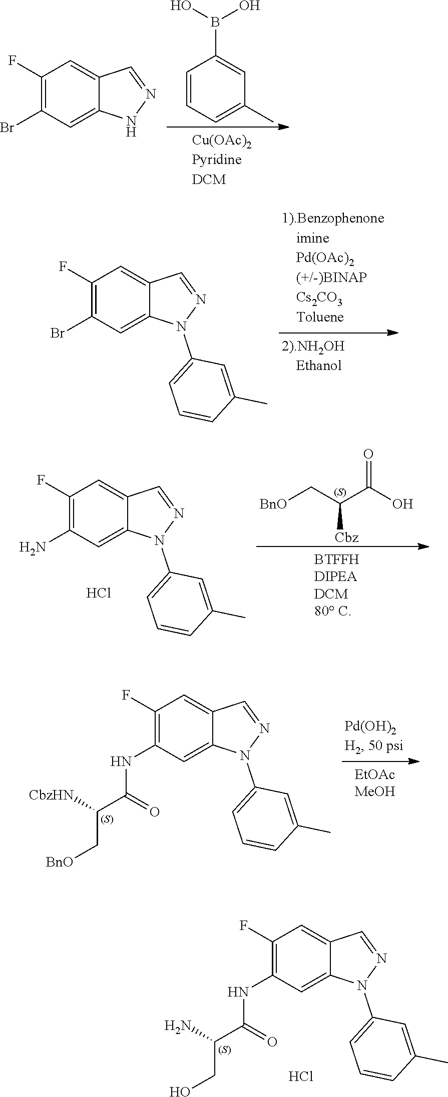

Step 1: 6-bromo-5-fluoro-1-(m-tolyl)-1H-indazole (2.2 g, 9.18 mmol), benzophenone imine (1.6 g, 8.83 mmol), Pd(OAc)₂ (160 mg, 0.71 mmol), (+/−)-BINAP (220 mg, 0.35 mmol), Cs₂CO₃ (4.7 g, 14.43 mmol) and toluene (35 mL) were combined in a glass bomb. The reaction mixture was bubbled with argon for 10 min and then the vessel sealed. The reaction mixture was heated to 100° C. for 15 h. The reaction mixture was diluted with EtOAc (25 mL) and filtered through a bed of celite. The celite bed was rinsed with an additional 25 mL of EtOAc. The filtrate was concentrated to dryness. LC/MS indicates that the desired product was formed. No further purification attempted. Yield not calculated at this step.

Step 2: In round bottom flask, was combined 6-bromo-5-fluoro-1H-indazole (2.0 g, 9.34 mmol), m-tolylboronic acid (1.5 g, 11.03 mmol), Cu(OAc)₂ (2.5 g, 13.76 mmol), and pyridine (1.6 mL, 19.82 mmol) in DCM (50 mL). The resultant suspension was stirred for 14 h. The reaction mixture was concentrated to dryness and then resuspended in a saturated NH₄Cl solution. The suspension was stirred for 5 min, EtOAc was added, and the mixture was stirred for an additional 45 min. The biphasic mixture was then filtered through a thin bed of celite. The filtrate was transferred to a separatory funnel and the layers were separated. The organic layer was dried over Na₂SO₄, filtered, and concentrated to dryness to give a crude yellow solid. LC/MS indicates the desired product. No further purification attempted at this stage.

Step 3: To a suspension of crude N-(diphenylmethylene)-5-fluoro-1-(m-tolyl)-1H-indazole-6-amine (2.9 g, 7.15 mmol, assumed) in EtOH (20 mL) was added a 50% aqueous hydroxylamine solution (4.7 mL, 23.01 mmol). The mixture was stirred for 19 h, at which point LC/MS indicated the desired amine product was formed. The mixture was concentrated to dryness, suspended in EtOAc (35 mL) and rinsed with DI H₂O (35 mL). The organic layer was dried over Na₂SO₄, filtered, and concentrated to give a crude oil. The crude was purified by CombiFlash chromatography (20-40% EtOAc:heptane) to give the desired freebase. The freebase was dissolved in EtOAc, stirred, treated with 2 N ethereal HCl, and filtered to give 5-fluoro-1-(m-tolyl)-1H-indazole-6-amine as a pale purple solid (650 mg, 33% yield over two steps). LC/MS and NMR are consistent.

Step 4: In a small glass bomb, were combined 5-fluoro-1-(m-tolyl)-1H-indazol-6-amine (150 mg, 0.38 mmol), (S)-3-(benzyloxy)-2-((benzyloxy)methyl)-3-oxopropanoic acid (290 mg, 0.92 mmol), fluoro-N,N,N′,N′-bis(tetramethylene)formamidinium hexafluorophosphate (290 mg, 0.92 mmol), DIPEA (2.2 mL, 12.65 mmol), and DCM (20 mL). The reaction vessel was sealed and heated to 80° C. for 48 h. LC/MS indicated the desired compound had been formed. The reaction mixture was diluted with water (25 mL) and additional DCM (10 mL) and they layers were separated. The organic layer was dried over Na₂SO₄, filtered, and concentrated. The resultant crude material was purified by chromatography (30% EtOAc:heptane) to give an inseparable mixture of a small amount starting amine and the desired benzyl (S)-(3-(benzyloxy)-1-((5-fluoro-1-(m-tolyl)-1H-indazol-6-yl)amino)-1-oxopropan-2-yl)carbamate (270 mg, yield not calculated at this stage). The material was taken on to the deprotection without further purification.

Step 5: To a solution of benzyl (S)-(3-(benzyloxy)-1-((5-fluoro-1-(m-tolyl)-1H-indazol-6-yl)amino)-1-oxopropan-2-yl)carbamate (270 mg, 0.48 mmol) in 10:1 EtOAc:MeOH (15 mL) was added Pd(OH)₂ (10 mg). The reaction vessel was sealed and pressurized to 60 psi with H₂. The reaction mixture was stirred under pressure for 18 h. At which point LC/MS indicated a complete deprotection. The reaction mixture was concentrated to dryness and then the crude purified by chromatography (10% MeOH:DCM). The freebase was dissolved in EtOAc (1 mL with a drop of MeOH for solubility). The solution was treated with 2 N ethereal HCl and the resultant suspension was stirred for 20 min. The suspension was filtered and dried under vacuum to give (S)-2-amino-N-(5-fluoro-1-(m-tolyl)-1H-indazol-6-yl)-3-hydroxypropanamidehydrochloride as an yellow solid (20 mg, 10% yield over two steps). NMR and LC/MS are consistent.

### Preparation of 3-(difluoromethyl)-1-(m-tolyl)-1H-indazol-6-amine hydrochloride

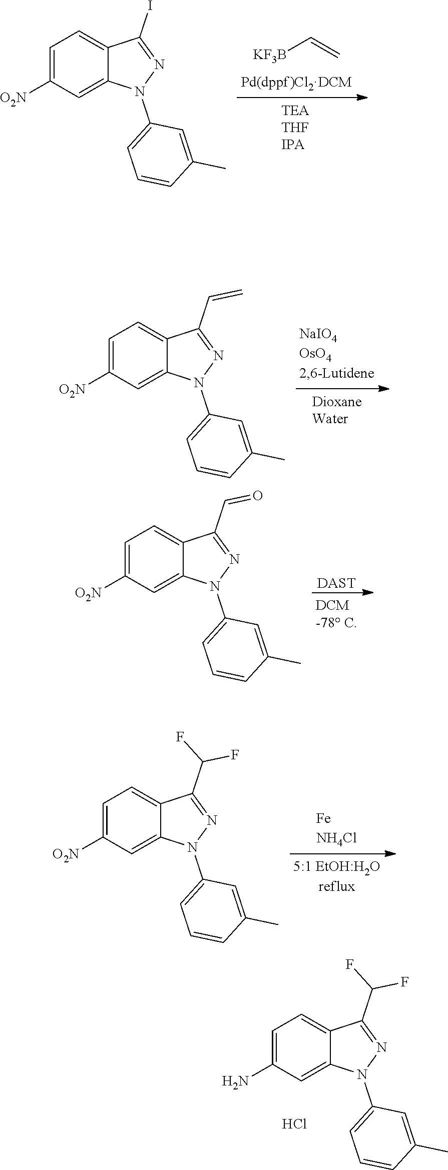

Step 1: A suspension of 3-iodo-6-nitro-1-(m-tolyl)-1H-indazole (4.0 g, 10.55 mmol) and potassium trifluoro(vinyl)borate (4.2 g, 31.35 mmol) in 4:1 IPA:THF (50 mL) was bubbled with N₂ for 20 min. TEA (10.0 mL, 71.75 mmol) and Pd(dppf)Cl₂DCM (400 mg) were added. The reaction vessel was sealed and heated to 100° C. for 3.5 h. LC/MS showed the complete consumption of the lodo compound. The reaction mixture was diluted with EtOAc and filtered through celite. The filtrate was concentrated to give a crude reddish brown solid. The crude was purified by CombiFlash chromatography (5% EtOAc:heptane) to give 6-nitro-1-(m-tolyl)-3-vinyl-1H-indazole as a yellow solid (2.6 g, 90% yield). NMR is consistent.

Step 2: To a suspension of 6-nitro-1-(m-tolyl)-3-vinyl-1H-indazole (2.6 g, 9.31 mmol), NaIO₄ (8.0 g, 37.40 mmol), and 2,6-lutidene (2.0 g, 18.67 mmol) in 3:1 dioxane:water (32 mL) was added a 4% aqueous solution of OsO₄ (4 drops). The reaction mixture was stirred for 16 h at which point TLC indicated the alkene compound was consumed. The mixture was diluted with DCM (150 mL) and rinsed with a brine solution (4×75 mL). The organic layer was dried over Na₂SO₄, filtered, and concentrated. The crude material was purified by CombiFlash chromatography (30% EtOAc:heptane) to give 6-nitro-1-(m-tolyl)-1H-indazole-3-carbaldehyde as a yellow solid (1.3 g, 50% yield). NMR is consistent.

Step 3: To a solution of 6-nitro-1-(m-tolyl)-1H-indazole-3-carbaldehyde (1.0 g, 3.56 mmol) in DCM (10 mL) at −78° C. was added DAST (860 mg, 5.34 mmol). The reaction mixture was stirred for 18 h slowly coming to room temperature. The solution was poured over an ice cold, saturated solution of NaHCO₃ and the mixture was stirred for 15 min. TLC indicated the starting aldehyde compound was consumed and the TLC matched the standard for the previous lot. The layers were separated and the organic layer was rinsed with a brine solution (2×50 mL). The organic layer was dried over Na₂SO₄, filtered, and concentrated to dryness. The crude material (1.1 g) was taken forward to the nitro reduction without further purification. Yield not calculated at this step.

Step 4: To a suspension of crude 3-(difluoromethyl)-6-nitro-1-(m-tolyl)-1H-indazole (1.1 g, 3.63 mmol) in 5:1 ethanol:water (24 mL) was added Fe powder (610 mg, 10.92 mmol) and NH₄Cl (90 mg, 1.68 mmol). The reaction mixture was refluxed for 3 h at which point LC/MS showed a complete conversion to the amine. The reaction was filtered through a bed of celite. The celite cake was rinsed with EtOAc (30 mL). The filtrate was concentrated to near dryness. The mixture was partitioned between EtOAc (50 mL) and water (50 mL). The organic layer was dried over Na₂SO₄, filtered, and concentrated to give a crude oil. The crude was purified by CombiFlash chromatography (20% EtOAc:heptane) to give the desired amine freebase. The crude was purified by CombiFlash chromatography (30% EtOAc:heptane) to give the desired amine freebase. The freebase was dissolved in EtOAc, treated with 2 N ethereal HCl, stirred for 30 min, and then filtered to give 3-(difluoromethyl)-1-(m-tolyl)-1H-indazol-6-amine hydrochloride as a yellow solid (580 mg, 53% yield over two steps). NMR and LC/MS are clean and consistent.

### Preparation of Compound 48

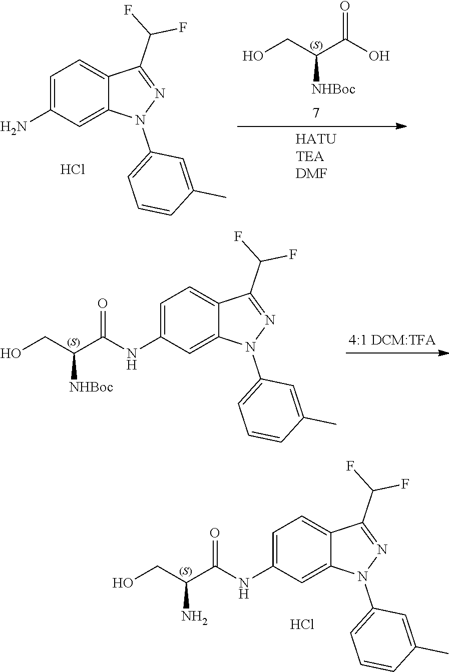

Step 1: To a solution of 3-(difluoromethyl)-1-(m-tolyl)-1H-indazol-6-amine hydrochloride (120 mg, 0.39 mmol) and HATU (190 mg, 0.50 mmol) in DMF (5 mL) at 0° C. was added a solution of Boc-L-serine (100 mg, 0.49 mmol) and TEA (135 μL, 0.97 mmol) in DMF (5 mL) dropwise over 3 min. The solution was stirred for 18 h, slowly coming to room temperature. LC/MS indicated a clean conversion. The reaction mixture was diluted with a saturated NaHCO₃ solution (20 mL) and extracted into EtOAc (20 mL). The organic layer was rinsed with a brine solution (3×10 mL). The organic layer was then dried over Na₂SO₄, filtered, and concentrated to give a crude amber oil. The crude was purified by CombiFlash chromatography (50% EtOAc:heptane) to give (S)-tert-butyl (3-hydroxy-1-oxo-1-((1-(4-fluoro-3-methyl)-1H-indazol-6-yl)amino)propan-2-yl)carbamate as an off-white semi solid (140 mg, 79% yield). LC/MS of the product is consistent. NMR was not taken at this stage.

Step 2: To a solution of (S)-tert-butyl (1-((3-(difluoromethyl)-1-(m-tolyl)-1H-indazol-6-yl)amino)-3-hydroxy-1-oxopropan-2-yl)carbamate (140 mg, 0.33 mmol) in DCM (4 mL) at 0° C. was added TFA (1 mL). The mixture was stirred for 3 h, slowly coming to room temperature. LC/MS indicated a complete deprotection. The reaction solution was concentrated to dryness and MTBE was added to the reaction vial. The mixture was treated with 2 N ethereal HCl and the resultant suspension was stirred for 30 min and filtered to give (S)-2-amino-N-(1-((3-(difluoromethyl)-1-(m-tolyl)-1H-indazol-6-yl)-3-hydroxypropanamide hydrochloride as a pale orange solid (100 mg, 77%). The solid was further dried in a vacuum oven at 50° C. for 48 h. LC/MS and NMR are consistent.

### Screening for KHK Inhibition

Specific coupled enzyme screening assays were developed for KHK-C and KHK-A using recombinant proteins. Purified human recombinant KHK-C and KHK-A were produced using by expression in E. coli BL-21 (DE3) using IPTG induction as a His-tagged fusion proteins and purified using Ni-NTA chromatography on His-Trap FF columns. For KHK-C, the coding region from NCBI refseq number NM_006488 was inserted at the Nde I site of pET28a(+) vector, which puts it downstream from an amino-terminal (His)₆ tag and a thrombin cleavage site. The same was done for KHK-A using the coding regions from NCBI refseq number NM_000221.2. The protein was expressed in BL-21 (DE3) using IPTG induction and purified using Ni-NTA (GE His Trap FF) column. The protein was washed with 100 column volumes, eluted with 3 column volumes, and dialyzed with HEPES, pH 7.4 buffers containing 20, 500, and 0 mM imidazole, respectively. The dialysate was concentrated by ultrafiltration (10kD MWCO) to >15 mg/mL protein. The protein prep was >95% pure on SDS-PAGE and the molecular weight was confirmed by relative mobility to be 33,730 Da (34,826 expected).

The assay uses the product of the KHK reaction, ADP, to drive a signal measured in real time for measuring the reaction rate. This consists of a 3-step, coupled-enzyme process by which pyruvate kinase (PK), which converts the ADP from the KHK reaction along with phosphoenolpyruvate (PEP) to ATP and pyruvate, respectively. The pyruvate is converted along with NADH to lactate and NAD+ by lactate dehydrogenase (LDH). The disappearance of NADH is measured kinetically by monitoring A₃₄₀ at 25° C. for 15 minutes. The enzymatic assay was carried out in a total reaction volume of 200 μL containing 33 mM triethanolamine-HCl, pH 7.4, 6 mM MgCl₂, 100 mM KCl, 0.1 mM ATP, 1.33 mM PEP, 0.3 mM NADH, 0.2-1.0 U of PK, 0.2-1.0 U of LDH, and 150-240 nM KHK-C or KHK-A (0.01-0.02 U). Fructose was added to initiate the reactions to 0.2 mM, except for the no fructose controls which utilized water. This high-throughput assay was used to identify inhibitors that have an IC₅₀ value<1 μM for KHK-C and to confirm that those values for KHK-A were not more than 2-fold lower.

### REFERENCES

The teachings of the following references, which are not admitted to be prior art by inclusion in this section, are incorporated herein by reference in their entirety to the extent that they are not inconsistent with the teachings herein.
1. Van den Berghe G, Bronfman M, Vanneste R, Hers H G. The mechanism of adenosine triphosphate depletion in the liver after a load of fructose. A kinetic study of liver adenylate deaminase. Biochem J. 1977; 162(3):601-609.
2. Ishimoto, T., M. A. Lanaspa, M. T. Le, et al. 2012. Opposing effects of fructokinase C and A isoforms on fructose-induced metabolic syndrome in mice. Proc Natl Acad Sci USA. 109: 4320-4325.
3. Li, X., X. Qian, L. X. Peng, et al. 2016. A splicing switch from ketohexokinase-C to ketohexokinase-A drives hepatocellular carcinoma formation. Nat Cell Biol. 18: 561-571.
4. Lanaspa, M. A., T. Ishimoto, N. Li, et al. 2013. Endogenous fructose production and metabolism in the liver contributes to the development of metabolic syndrome. Nat Commun. 4: 2434.
5. Lanaspa, M. A., T. Ishimoto, C. Cicerchi, et al. 2014. Endogenous fructose production and fructokinase activation mediate renal injury in diabetic nephropathy. J Am Soc Nephrol. 25: 2526-2538.
6. Lanaspa M A. Kuwabara M, Andres-Hernando A, Li N, Cicerchi C, Jensen J, Orlicky D J, Roncal-Jimenez C, Ishimoto T, Nakagawa T, Rodriguez-Iturbe B, MacLean P S, Johnson R J. High Salt Intake Causes Leptin Resistance and Obesity in Mice by Stimulating Endogenous Fructose Production and Metabolism Proc Natl Acad Sci USA PNAS Mar. 5, 2018. 201713837
7. Lanaspa M A, Andres-Hernando A, Orlicky D J, Cicerchi C, Jang C, Li N, Milagres T, Kuwabara M, Wempe M F, Rabinowitz J D, Johnson R J, Tolan D R. Ketohexokinase C blockade ameliorates fructose-induced metabolic dysfunction in fructose-sensitive mice. J Clin Invest (in press)
8. Andres-Hernando A, Li N, Cicerchi C, Inaba S, Chen W, Roncal-Jimenez C, Le M T, Wempe M F, Milagres T, Ishimoto T, Fini M, Nakagawa T, Johnson R J, Lanaspa M A. Protective role of fructokinase blockade in the pathogenesis of acute kidney injury in mice Nat Commun. 2017 Feb. 13; 8:14181
9. Roncal Jimenez, C. A., T. Ishimoto, M. A. Lanaspa, et al. 2014. Fructokinase activity mediates dehydration-induced renal injury. Kidney Int. 86: 294-302.
10. Mirtschink P, Krishnan J, Grimm F, et al. 2015. HIF-driven SF3B1 induces KHK-C to enforce fructolysis and heart disease. Nature. 522: 444-449.
11. Admyre T, Amrot-Fors L, Andersson M, Bauer M, Bjursell M, Drmota T, Hallen S, Hartleib-Geschwindner J, Lindmark B, Liu J, Löfgren L, Rohman M, Selmi N, Wallenius K. Inhibition of AMP deaminase activity does not improve glucose control in rodent models of insulin resistance or diabetes Chem Biol 2014 Nov. 20; 21(11):1486-96. doi: 10.1016/j.chembiol.2014.09.011.
12. Oppelt S A, Sennott E M, Tolan D R. Aldolase-B knockout in mice phenocopies hereditary fructose intolerance in humans. Mol Genet Metab. 2015; 114(3):445-450.
13. U.S. Pat. No. 6,894,047.
14. U.S. Pat. No. 6,570,013.
15. U.S. Pat. No. 6,294,538.
16. US Published Patent Application No. 20050020578.
17. US Patent Pub. 20110263559.
18. Adelman R C, Ballard F J, Weinhouse S. Purification and properties of rat liver fructokinase. J Biol Chem. 1967; 242(14):3360-3365.
19. Asipu A, Hayward B E, O'Reilly J, Bonthron D T. Properties of normal and mutant recombinant human ketohexokinases and implications for the pathogenesis of essential fructosuria. Diabetes. 2003; 52(9):2426-2432.
20. Zhang J H, Chung T D, Oldenburg K R. A Simple Statistical Parameter for Use in Evaluation and Validation of High Throughput Screening Assays. J Biomol Screen. 1999; 4(2):67-73.
21. Bais R, James H M, Rofe A M, Conyers R A. The purification and properties of human liver ketohexokinase. A role for ketohexokinase and fructose-bisphosphate aldolase in the metabolic production of oxalate from xylitol. Biochem J. 1985; 230(1):53-60.
22. Le M T, Lanaspa M A, Cicerchi C M, et al: Bioactivity-Guided Identification of Botanical Inhibitors of Ketohexokinase. PLoS One 11:e0157458, 2016.
23. Thurston J H, Jones E M, Hauhart R E. Decrease and inhibition of liver glycogen phosphorylase after fructose. An experimental model for the study of hereditary fructose intolerance. Diabetes. 1974; 23(7):597-604.
24. Van den Berghe G, Hue L, Hers H G. Effect of the administration of fructose on the glycogenolytic action of glycogen. Biochem J. 1973; 134:637.
25. Roncal Jimenez, C. A., T. Ishimoto, M. A. Lanaspa, et al. 2014. Fructokinase activity mediates dehydration-induced renal injury. Kidney Int. 86: 294-302.
26. Zhang, X., F. Song, G. H. Kuo, et al. 2011. Optimization of a pyrazole hit from FBDD into a novel series of indazoles as ketohexokinase inhibitors. Bioorg Med Chem Lett. 21: 4762-4767.
27. “Design of Prodrugs”, ed. H. Bundgaard, Elsevier, 1985.
28. Handbook of Pharmaceutical Excipients, published by the American Pharmaceutical Association and the Pharmaceutical Society of Great Britain.
29. Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.
30. US 20130224218, entitled Methods and Compositions for the Inhibition of Fructokinase.
31. WO2012019188, entitled Methods and Compositions for the Inhibition of Fructokinase.
32. WO2018170517, entitled Indazole Inhibitors of Fructokinase (KHK) and Methods of Use in Treating KHK-Mediated Disorders or Diseases.”

1. A compound according to Formula (I): 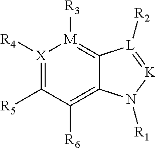
or a pharmaceutically acceptable salt, prodrug, or stereoisomer thereof,
wherein
M, L, K, and X are each independently=carbon or nitrogen;
R1=hydrogen, halogen, alkyl, ether, aryl, heteroaryl, cyclo, or heterocyclo,
R2=hydrogen, deuterium, halogen, cyano, alkyl, ether, cyclo, or heterocyclo when L=C or R2 is not present when L=nitrogen;
R3=hydrogen, halogen, deuterium, hydroxy, amino, alkyl, ether, alkoxy, cyano, cyclo, or heterocyclo when M=C or R3 is not present when M=nitrogen, wherein, when n=1 and m=1, R3=fluoro;
R4 and R6 are each independently=hydrogen, deuterium, halogen, alkyl, ether, cyclo, or heterocyclo;
R5=a substitution according to Formula (IIa) or (IIb): 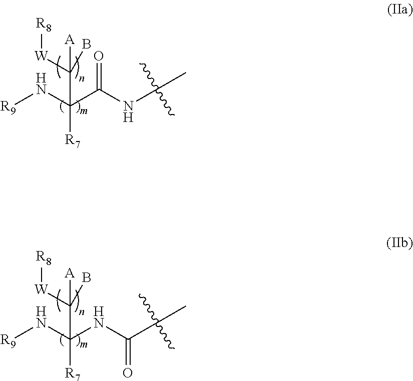
wherein R7 is selected from H, D, O-lower alkyl, or lower alkyl;
A and B are independently H, D, CH₃, CH₂CH₃, or cycloalkyl, or collectively define a carbonyl group, or are joined to form a 3-6 membered ring with a heteroatom or a heterofunctional group selected from O, NH, N-lower alkyl, S, or SO₂;
W is selected from CH₂, CHR7, C(R7)₂, O, or NH;
R8 is selected from H, —COAlkyl, —COAryl, —COOAlkyl, —COOAryl, —CONHAlkyl, or —CONHAryl;
R9 is selected from H, lower alkyl, —COOAlkyl, or —COOAryl;
n is 0, 1, 2, or 3;
m independently of n is 1, 2, or 3, wherein, when n=0, m>1;
wherein when K=carbon, then R1=m-tolyl or p-fluoro, R3=hydrogen or fluoro, and R5=serine,
wherein R7 and R9 are optionally joined to form a 4-7 membered ring, and wherein, when present, the 4-7 membered ring optionally comprises 0-5 methyl groups, OMethyl, or OR8; and
wherein, when m>1 and W=CH₂, W and R7 are optionally joined to form a 3-6 membered carbocyclic or heterocyclic ring comprising O or R9.

2. The compound of claim 1, wherein R1 comprises: 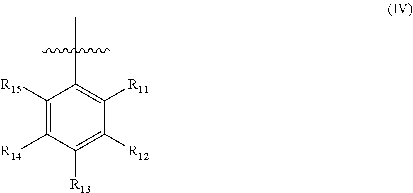
and wherein each of R11, R12, R13, R14, and R15 is independently selected from the group consisting of hydrogen, deuterium, halogen, hydroxy, a carboxylic acid moeity, amido, alkyl, aryl, heteroaryl, cyclo, heterocyclo, alkoxy, haloalkyl, haloalkoxy, and carboxylamido.

3. The compound of claim 2, wherein each of R11, R12, R13, R14, and R15 is independently selected from the group consisting of —H, -D, —F, —Cl, Br, —CF₃, —CF₂H, —CFH₂, —OMe, —OCF₃, —C(O)NH₂, and —CH₃.

4. The compound of claim 1, wherein R1 comprises a member selected from the group consisting of:

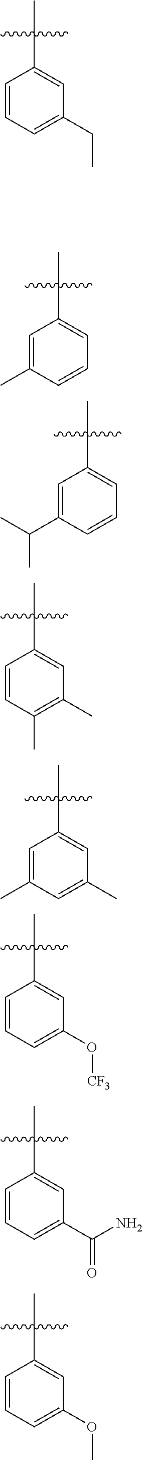

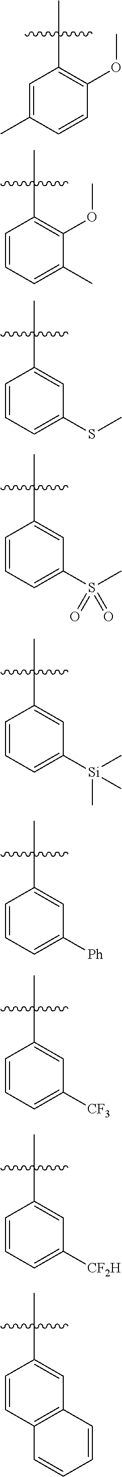

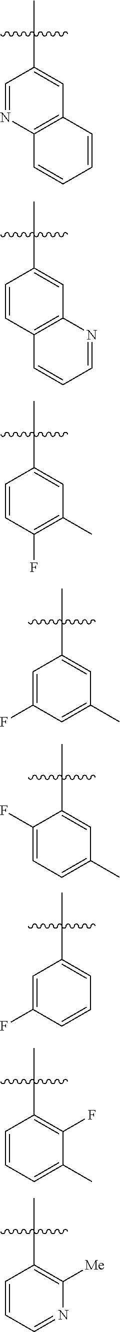

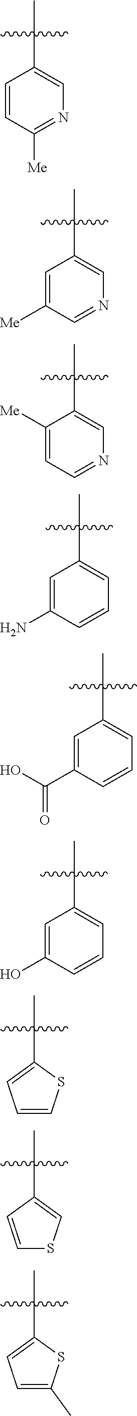

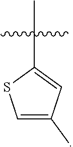

5. The compound of claim 1, wherein

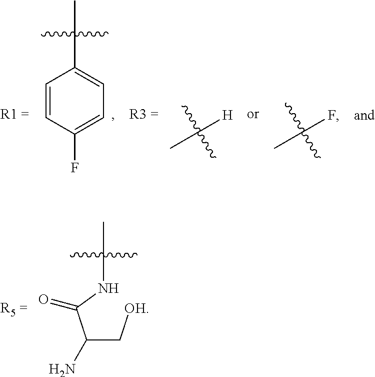

6. The compound of claim 1, wherein R3=F and

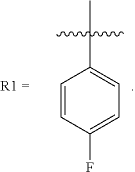 [SMILES: *c1ccc(F)cc1]

7. The compound of claim 1, wherein L=C and R2 is selected from a member of the group consisting of:

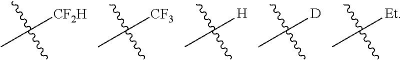

8. The compound of claim 1, wherein L=C and R3 is selected from a member of the group consisting of:
—CH₃,

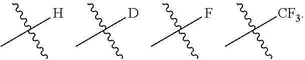

9. The compound of claim 1, wherein M=nitrogen.

10. The compound of claim 1, wherein R4 and R6 are independently selected from a member from the group consisting of:

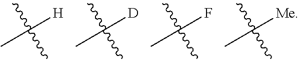

11. The compound of claim 1, wherein R5 comprises: 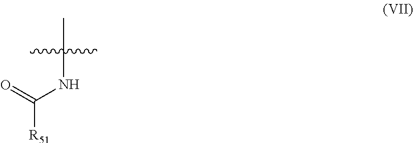
where R51=hydrogen, halogen, alkyl, ether, aryl, heteroaryl, cyclo, or heterocyclo;
or 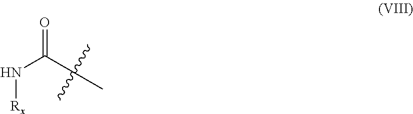
where R51=hydrogen, halogen, alkyl, ether, aryl, heteroaryl, cyclo, or heterocyclo.

12. The compound of claim 1, wherein R5 comprises: 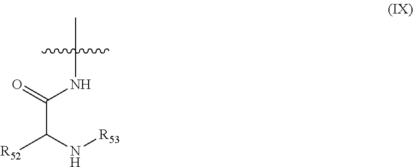
where R52 and R53 are independently hydrogen, halogen, alkyl, ether, aryl, heteroaryl, cyclo, or heterocyclo.

13. The compound of claim 1, wherein R5 comprises: 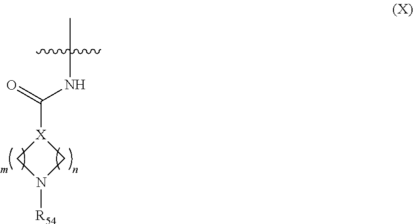
where n and m are independently=0 to 3
X of Formula (X)=C or N, and
R54=hydrogen, halogen, alkyl, ether, aryl, heteroaryl, cyclo, or heterocyclo.

14. The compound of claim 1, wherein R5 comprises: 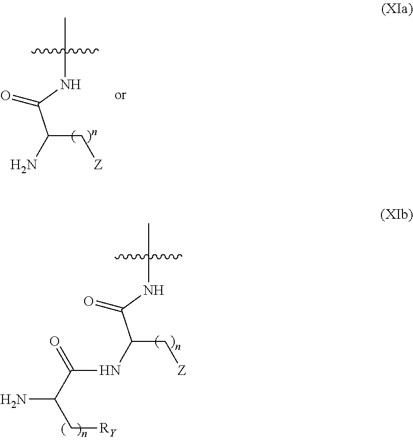
where n=0 to 5,
Z=alkyl, hydroxyl, —B(OH)₂, cyano, carboxyl, an amino acid functional group, alkylamino, alkylcarboxy, heterocyclic, aryl, heteroaryl, alkyl sulfide, thiol, or alkylurea; and
R_{Y}=alkyl, hydroxyl, —B(OH)₂, cyano, carboxyl, an amino acid functional group, alkylamino, alkylcarboxy, heterocyclic, aryl, heteroaryl, alkyl sulfide, thiol, or alkylurea.

15. The compound of claim 1, wherein R5 comprises: 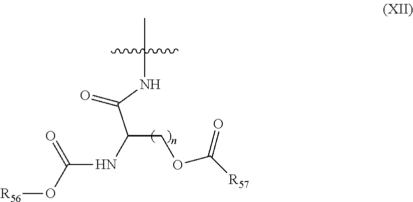
where n=0 to 5, and
R56 and R57 are independently hydrogen, aryl, alkyl, or alkenyl.

16. The compound of claim 1, wherein R5 comprises: 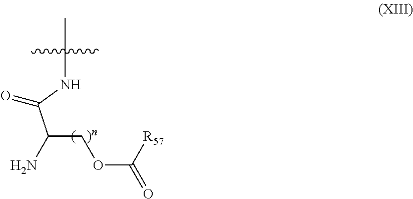
where n=0 to 5, and
R57 is hydrogen, alkyl, alkenyl, aryl, heteroaryl, cyclo, or heterocyclo.

17. The compound of claim 1, wherein R5 comprises: 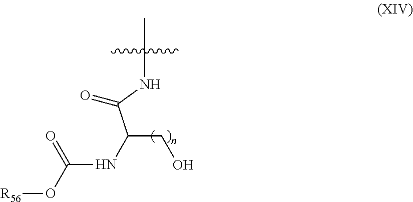
where n=0 to 5, and
R56 of Formula (XIV) is hydrogen, aryl, heteroaryl, alkyl, ester, or alkenyl.

18. The compound of claim 1, wherein R5 comprises: 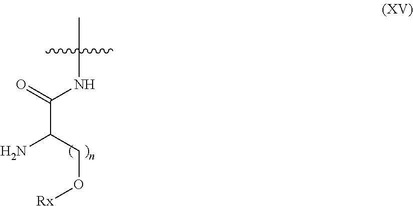
where n=0 to 5 and Rx=phosphate or phosphonate.

19. The compound of claim 18, wherein Rx=phosphate, wherein the phosphate comprises one of: 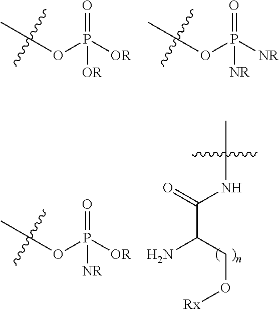
wherein R=hydrogen, alkyl, or alkenyl and n=0 to 5.

20. The compound of claim 1, wherein R5 comprises a member from the group consisting of:

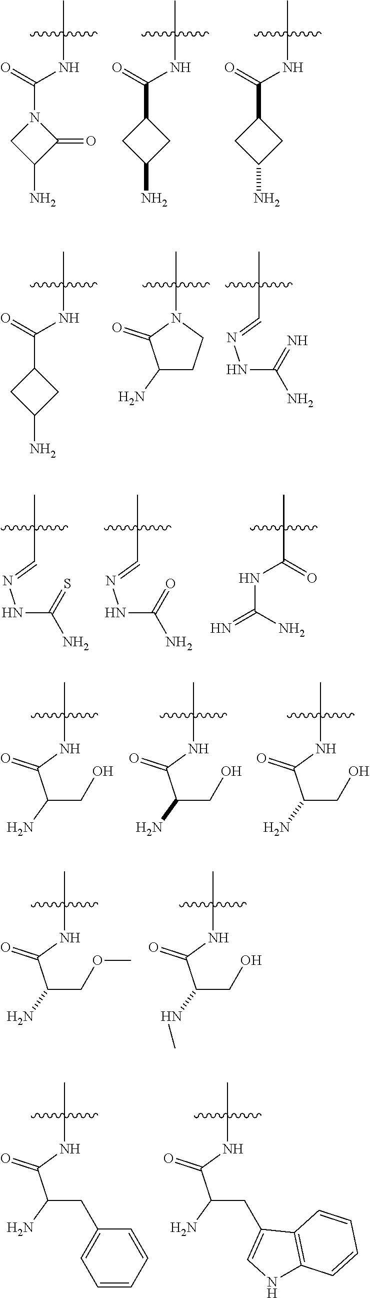

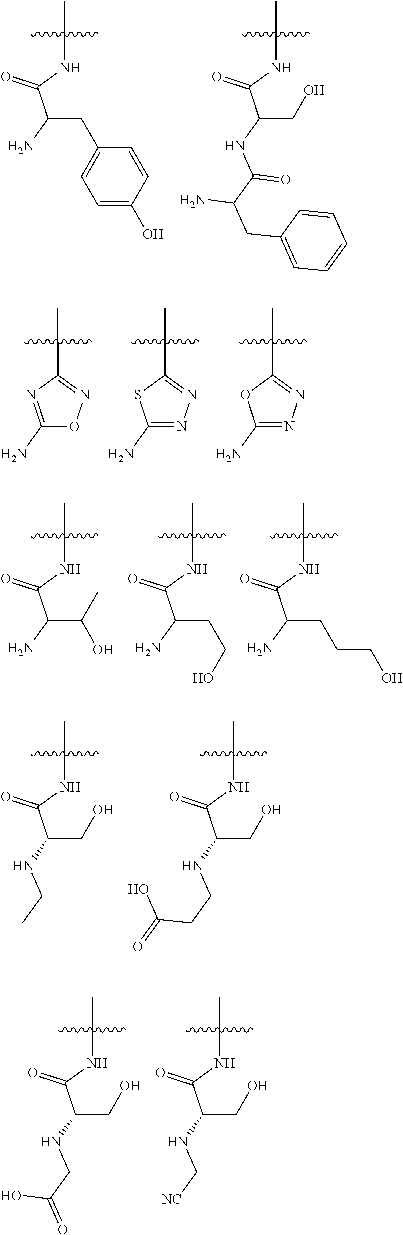

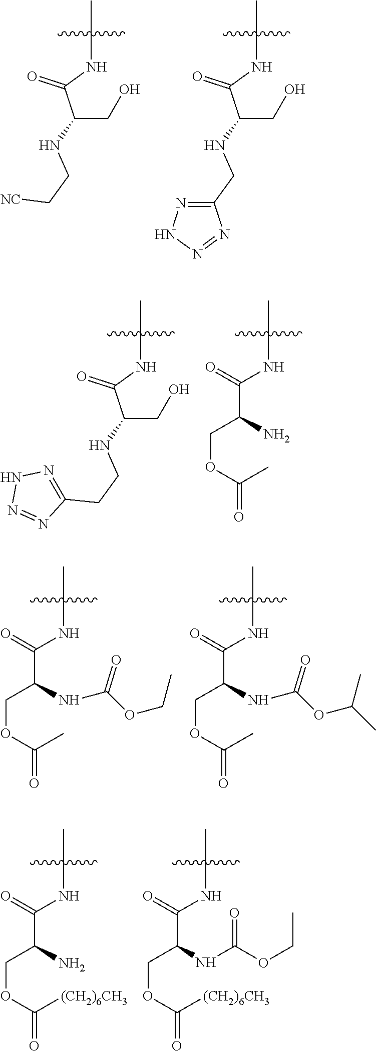

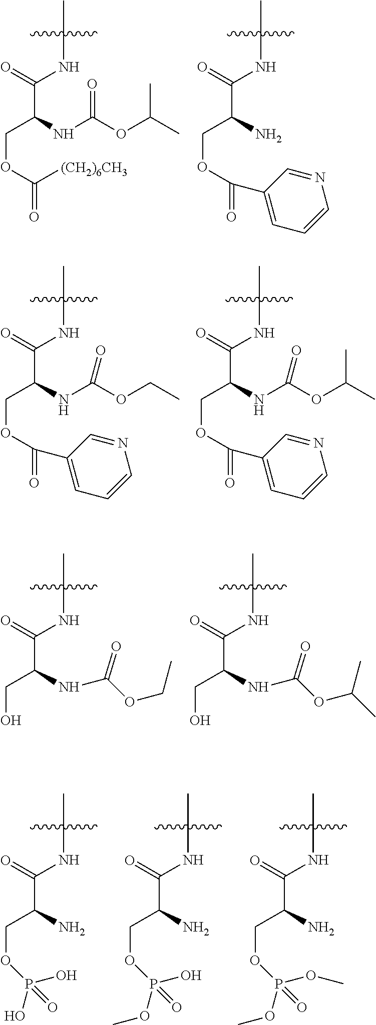

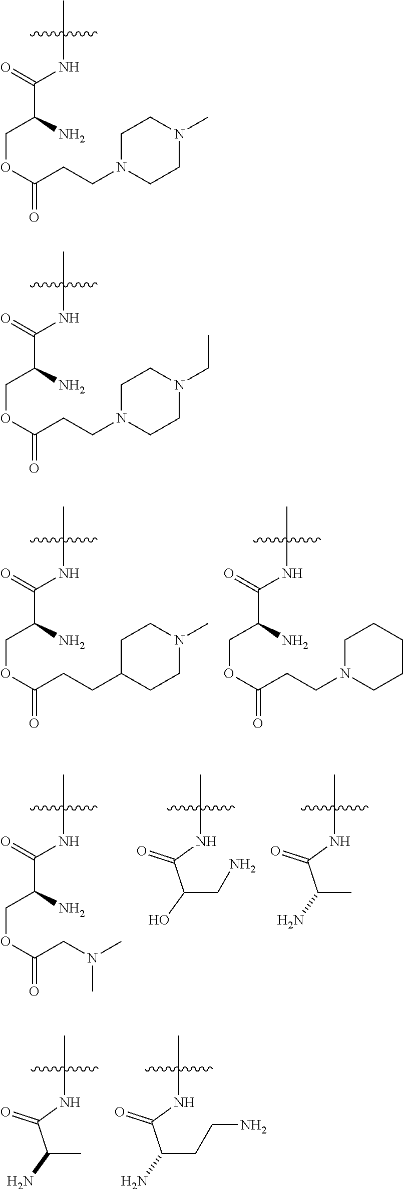

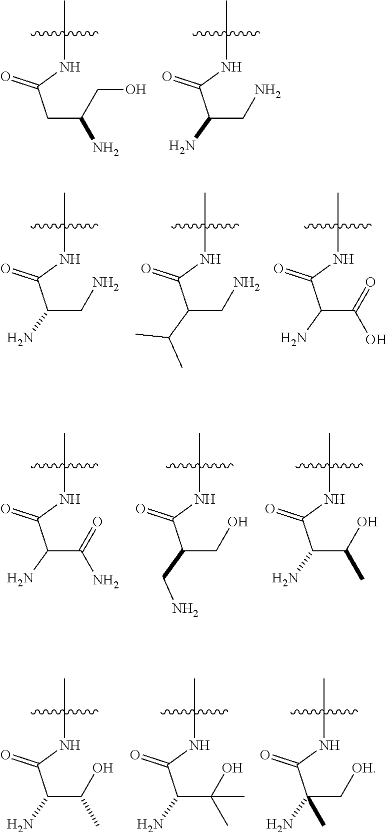

21. The compound of claim 1, wherein K=carbon.

22. The compound of claim 1, wherein m>1 and W=CH₂, and W and R7 are joined to form a 3-6 membered carbocyclic or heterocyclic ring comprising O or R9.

23. The compound of claim 1, wherein R7 and R9 are joined to form a 4-7 membered ring, and the 4-7 membered ring comprises 0-5 methyl groups, OMe, or OR8.

24. The compound of claim 1, wherein the compound comprises a prodrug of a compound according to Formula (I).

25. The compound of claim 1, wherein the compound comprises a compound as set forth in Table 1.

26. The compound of claim 1, wherein the compound comprises a formula according to (Vb):

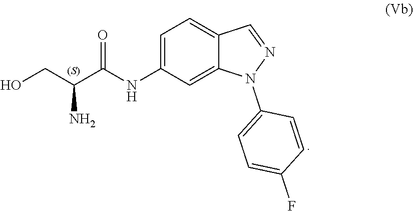

27. A pharmaceutical composition comprising one or more compounds according to claim 1 and a pharmaceutically acceptable carrier.

28. The pharmaceutical composition of claim 27, further comprising at least one therapeutic conjunctive compound.

29. The pharmaceutical composition of claim 28, wherein the at least one therapeutic conjunctive compound is at least one selected from the group consisting of an angiotensin-converting enzyme (ACE) inhibitor, an aldosterone antagonist, an amphetamine, an amphetamine-like agent, an Angiotensin II receptor antagonist, an anti-oxidant, an aldose reductase inhibitor, a biguanide, a sorbitol dehydrogenase inhibitor, a thiazolidinedione, a glifozin, a glitazone, a thiazide diuretic, a thiazide-like diuretic, a triglyceride synthesis inhibitor, and an uric acid lowering agent.

30. A pharmaceutical composition produced by a process comprising mixing a compound of claim 1 and a pharmaceutically acceptable carrier.

31. A process for making a pharmaceutical composition, the process comprising mixing a compound according to claim 1 and a pharmaceutically acceptable carrier.

32. A method of treating or preventing a disorder or disease mediated by ketohexokinase, the method comprising administering a therapeutically effective amount of the compound according to claim 1.

33. The method of claim 32, further comprising a pharmaceutically acceptable carrier.

34. The method of claim 32, wherein the disorder or disease mediated by ketohexokinase is selected from the group consisting of obesity, elevated glucose levels, glucose intolerance, insulin resistance, Type I diabetes mellitus, Type II diabetes mellitus, Metabolic Syndrome, elevated triglycerides, hyperlipidemia, and hypertension.

35. The method of claim 32, wherein the disorder or disease mediated by ketohexokinase is selected from the group consisting of nonalcoholic or alcoholic liver disease, obesity, Type II diabetes mellitus, and Metabolic Syndrome.

36. The method of claim 32, wherein the disorder or disease mediated by ketohexokinase is selected from the group consisting of colon cancer, liver cancer or liver metastases, glioma, breast cancer, lung cancer, acute myelogenous leukemia, and pancreatic cancer.

37. The method of claim 32, wherein disorder or disease mediated by ketohexokinase is selected from the group consisting of Alzheimer's disease, alcohol addiction, and attention deficit hyperactivity disorder.
